(19) United States
(12) Patent Application Publication
(10) Pub. No.: US 2023/0390226 A1
(43) Pub. Date: Dec. 7, 2023

(54) INTRANASAL ADMINISTRATION OF SURAMIN FOR TREATING NERVOUS SYSTEM DISORDERS

(71) Applicant: PaxMedica, Inc., Tarrytown, NY (US)

(72) Inventors: Michael DERBY, Greenwich, CT (US); Zachary ROME, Nyack, NY (US)

(73) Assignee: PaxMedica, Inc., Tarrytown, NY (US)

(21) Appl. No.: 18/033,167

(22) PCT Filed: Oct. 20, 2021
(86) PCT No.: PCT/US21/55908

§ 371 Date: Apr. 21, 2023

(51) Int. Cl. A61K 31/17; A61P 25/28; A61K 9/00

(52) U.S. Cl. CPC A61K 31/17; A61P 25/28; A61K 9/0043

(57) ABSTRACT

The present invention provides methods and compositions for intranasally (IN) treating nervous system disorders such as cognitive, social, or behavioral disabilities, and neurodevelopmental disorders, More specifically, the present invention demonstrates that intranasal administration of suramin is effective to ameliorate or provide an improvement in one or more of the symptoms or manifestations associated with these disabilities and disorders.

### CROSS-REFERENCE TO RELATED APPLICATIONS

This patent application is a national phase entry under 35 U.S.C. § 371 of International Application Number PCT/US2021/055908, filed Oct. 20, 2021, which claims a benefit under 35 U.S.C. § 119(e) to the Oct. 22, 2020 filing date of U.S. Provisional Patent Application Ser. No. 63/104,350, all of which are herein incorporated by reference in their entirety.

### FIELD OF THE INVENTION

The present invention provides methods and compositions for treating nervous system disorders, including cognitive, social, or behavioral disabilities, neurodevelopmental disorders, psychiatric disorders, neurological disorders, and central nervous systems disorders. More specifically, the present invention provides methods and compositions for a nasal spray product for intranasally (IN) delivering a therapeutically effective amount of the antipurinergic agent, suramin, and pharmaceutically acceptable salts, esters, solvates, and prodrugs thereof, to treat or ameliorate the symptoms and manifestations associated with these disorders.

### BACKGROUND OF THE INVENTION

Nervous system disorders, whether mild or severe in their manifestation, affect many individuals in the US and around the world. These disorders have an impact beyond the individual patient and affect family members, caregivers, and society in general.

Nervous system disorders, include, cognitive, social, or behavioral disabilities, neurodevelopmental disorders, psychiatric disorders, neurologic disorders, and central nervous system (CNS) disorders. These nervous system disorders include, inter alia, autism spectrum disorder (ASD), fragile X syndrome (FXS), fragile X-associated tremor/ataxia syndrome (FXTAS), myalgic encephalomyelitis/chronic fatigue syndrome (ME/CFS), post-traumatic stress syndrome (PTSD), Tourette's syndrome (TS), Parkinson's Disease, Angelman syndrome (AS), and the CNS disorder manifestations often associated with Lyme disease and other tick-borne diseases, and the nervous system and central nervous system (CNS) disorders associated with COVID-19 and other viruses (e.g. Epstein Barr Human Herpesvirus 6 and 7, Herpes Simplex Virus, Cytomegalovirus, and others), including their long term effects. Note that this list of nervous system disorders is exemplary and that there are many others which can benefit from the present invention. Current treatments for these exemplified disorders are limited and often targeted to specific symptoms such as seizures, anxiety, depression, attention deficit/hyperactivity, sleep disorders, cognitive impairment, and the like. Even though there is much research in the area and the potential for new or known therapeutic agents for such treatments, it is not always apparent how to safely and effectively administer these agents and what the optimal dose and dosing regimens may be. It is demonstrated herein as set forth in the examples, that antipurinergic agents can be administered for treating these disorders according to a pharmacokinetic and pharmacodynamic treatment regimen that would not have been predicted a priori. These agents were administered at dosages and frequencies not previously disclosed or contemplated in the scientific literature, which led to the discovery of a dynamic, nonlinear correlation between efficacy and blood levels of the agent over time.

Autism is associated with a combination of genetic and environmental factors and has been reported to have an incidence in the US of about 1 in 60 children. Global prevalence estimates for autism are about 25 million individuals. Autism is also referred to as autism spectrum disorder (ASD), because it includes a broad range of symptoms characterized by challenges with social skills, repetitive behaviors, speech and nonverbal communication. In 2013, the American Psychiatric Association merged four distinct autism diagnoses into the single diagnosis of autism spectrum disorder. These diagnoses include autistic disorder, childhood disintegrative disorder, pervasive developmental disorder-not otherwise specified (PDD-NOS), and Asperger syndrome. Signs and symptoms of autism usually appear by age 2 or 3. Autism spectrum disorder is a condition related to brain development that impacts how a person perceives and socializes with others, causing problems in social interaction and communication. The disorder can also include limited and repetitive patterns of behavior.

Research shows that early intervention can lead to positive outcomes as described in the following references: Chaste P, Leboyer M (2012). “Autism risk factors: genes, environment, and gene-environment interactions”. Dialogues in Clinical Neuroscience. 14 (3): 281-92. PMC 3513682. PMID 23226953; and Centers for Disease Control and Prevention Morbidity and Mortality Weekly Report, Prevalence of Autism Spectrum Disorder Among Children Aged 8 Years—Autism and Developmental Disabilities Monitoring Network, 11 Sites, United States, 2014 Surveillance Summaries/Apr. 27, 2018/67 (6); 1-23.

There is currently no cure for autism spectrum disorder, and no US FDA approved medications to treat the core symptoms. According to the American Psychiatric Association's (APA's) Diagnostic and Statistical Manual of Mental Disorders (DSM-V) diagnostic criteria, the core symptoms of autism spectrum disorder include: persistent deficits in social-emotional reciprocity which results in difficulty developing, maintaining, and understanding relationships; deficits in verbal and nonverbal social communication; and restricted, repetitive patterns of behavior, interests or activities Persons with ASD often have many associated (i.e. non-core) symptoms including hyper- or hypo-reactivity to sensory input or unusual interest in sensory aspects of the environment, clinically significant impairment in social, occupational, or other important areas of current functioning, cognitive impairment, impulsiveness, attention deficit and hyperactivity symptoms, sleep disturbances, gastrointestinal complaints and food/chemical sensitivities, unusual eating habits, depression, mood disorders, anxiety, seizures, irritability, temper outbursts, sometimes violent behavior which can be self-directed or directed towards others.

Despite the prevalence of these core symptoms, instead, the focus of current therapies is on treating some of the accompanying non-core symptoms with various medications such as antipsychotics, anxiolytics, antidepressants, stimulants or medications for insomnia. Non-core symptoms that are often manifested include depression, seizures, anxiety, sleep disorders, hyperactivity, and trouble focusing. Also, behavioral, occupational, and speech therapies and other non-pharmacological interventions are employed. However, the exact causes of autism are not fully understood, thus contributing to the challenges of new drug development program.

Fragile X syndrome (FXS) is a rare, genetic neurodevelopmental disorder that affects approximately 1 in 4,000 people in the US. It is associated with highly variable cognitive and behavioral manifestations and has many overlapping features with ASD. It is an X-linked disorder, meaning that the genetic mutation occurs on the X chromosome. In FXS, there is a trinucleotide repeat expansion in the FMR1 gene. A trinucleotide expansion is a particular gene mutation in which a sequence of three nucleotide base pairs improperly repeats itself multiple times. In the case of FXS, the repeating trinucleotide sequence is cytosine-guanine-guanine (CGG). Normally, this DNA segment is repeated from 5 to about 40 times. In people with FXS, the segment is repeated more than 200 times. This typically results in no functional FMR1 mRNA transcript being produced, and the protein that is normally encoded by this transcript (fragile X mental retardation protein (FMRP)) is also absent.

Fragile X-associated tremor/Ataxia (FXTAS) is a different disorder, but genetically related to FXS. It is an “adult onset” rare, genetic neurodegenerative disorder, usually affecting males over 50 years of age. Females comprise only a small part of the FXTAS population, and their symptoms tend to be less severe. FXTAS affects the neurologic system and progresses at varying rates in different individuals.

FXS patients have the “full mutation” in the FMR1 gene (typically well over 200 CGG trinucleotide repeats), but patients with FXTAS are considered premutation ‘carriers’ of the FMR1 gene, as they have CGG trinucleotide repeats numbering in the range of 55-200. The function of the FMR1 gene is to make a protein (FMRP) that is important in brain development and for the maintenance and regulation of synaptic connections between neurons. Researchers believe that (for unknown reasons) having the premutation leads to the overproduction of FMR1 mRNA (which contains the expanded repeats). Researchers also suspect that the high levels of mRNA are what cause the signs and symptoms of FXTAS, but more research is needed to confirm these hypotheses.

Individuals with FXTAS usually experience symptoms after the age of 55. As premutation carriers age, especially men, the likelihood of experiencing symptoms rises. This likelihood reaches 75 percent by age 75 for premutation men. The progression of symptoms, including memory loss, slowed speech, tremors, and a shuffling gait, is gradual, with interference in daily activities by tremors and falls occurring around ten years after onset of the first symptoms. Dependence on a cane or walker occurs approximately 15 years after first exhibiting the symptoms of the disorder. Some people with FXTAS show a step-wise progression (i.e., symptoms plateau for a period of time but then suddenly get worse) with acute illnesses, major surgery, or other major life stressors causing symptoms to worsen more quickly.

The prevalence of FXTAS is unknown, although current estimates suggest that about 30%-40% of male FMR1 premutation carriers over 50 years of age, within families already known to have someone with Fragile X, will ultimately exhibit some features of FXTAS. There is no FDA approved therapy for FXTAS and currently used treatments only address the symptoms of the condition, rather than targeting the pathophysiology itself.

Myalgic encephalomyelitis/chronic fatigue syndrome (ME/CFS) can be debilitating. Chronic fatigue syndrome is also referred to as myalgic encephalomyelitis (ME) or the combined term myalgic encephalomyelitis/chronic fatigue syndrome (ME/CFS), which is a complex, variable symptom, fatiguing, long-term medical condition. ME/CFS can cause a worsening of symptoms after physical or mental activity referred to as post-exertional malaise (PEM). Patients with ME/CFS also often have sleep disturbances, joint and muscle pain, cognitive impairment, and significant orthostasis. Patients suffering from ME/CFS often have a greatly lowered functional ability to complete routine activities of daily living.

Post-traumatic stress disorder (PTSD) is classified as an anxiety disorder and can also be debilitating. PTSD can develop after a person is exposed to a traumatic event, such as warfare, sexual assault, or other significant traumatic event. PTSD symptoms can include hyperarousal, irritability, anger, depression, disturbing thoughts, feelings, dreams, or other intrusive recollections of the traumatic events, and also mental or physical distress to trauma-related cues. The symptoms of PTSD can be long lasting and result in significant functional impairment.

Tourette's syndrome (TS) is a neurodevelopmental disorder characterized by multiple movement, i.e. motor tics and at least one vocal, i.e. phonic tics. TS typically has onset in childhood or adolescence. The tics are typically preceded by an unwanted, uncontrollable urge or sensation in the affected muscles. Examples of these tics include blinking, coughing, throat clearing, sniffing, and facial movements. Although the exact cause is unknown, it is believed that TS involves a combination of genetic and environmental factors. More specifically there may be involvement of dysfunction in the neural circuits between the basal ganglia and related structures in the brain. At present there is no cure for TS. Haloperidol (Haldol), pimozide (Orap), and aripiprazole (Abilify) are currently the only medications approved by the U.S. Food and Drug Administration (FDA) to treat tics; however, these medications all have significant acute and long term side effects.

Parkinson's disease (PD) is a degenerative disorder of the nervous system that affects the motor system. The exact cause of the disease is unknown and may involve both genetic and environmental factors. The motor symptoms of PD include tremor, rigidity, slowness of movement, and difficulty with walking. These motor symptoms are also known as parkinsonism or parkinsonian syndrome. Also, cognitive, mood, and behavioral symptoms can be present including depression, anxiety, apathy, dementia, sleep disturbances, and sensory disturbances. The physical neurological changes associated with PD have been linked to the death of dopaminergic neurons in the substantia nigra, which is a region of the midbrain. This cell death is associated with a deficit of dopamine.

Angelman syndrome (AS), which is also known as Angelman's syndrome is a genetic disorder that affects the nervous system. Physical characteristics of the syndrome include microcephaly (i.e. a small head), In addition to physical characteristics such as a small head, telecanthus or dystopia canthorum (i.e., an increased distance between the inner corners of the eyelids), a wide mouth, and hands with tapered fingers, abnormal creases and broad thumbs The syndrome is associated with severe intellectual disability, developmental disability (e.g., a lack of functional speech), seizures (e.g. epileptic seizures), balance and movement problems, and sleep problems. Also, the electroencephalogram (EEG) of individuals with AS is usually abnormal. However, individuals with AS have a happy personality and are affectionate and seek human interaction. There is currently no cure available for AS. The seizures can be controlled by the use of one or more types of anticonvulsant medications. However, there are difficulties in ascertaining the levels and types of anticonvulsant medications needed to establish control, because people with AS often have multiple types of seizures.

Lyme disease (sometimes abbreviated LD) is an infectious disease caused by the bacteria Borrelia burgdorferi and Borrelia mayonii, carried primarily by black-legged or deer ticks. It is transmitted to the bloodstream by the bite of an infected ticks. The gram-negative bacterial species Borrelia burgdorferi, which can exist as a spirochete, is the major causative species for the disease. A common sign of a Lyme disease infection is an expanding red circular rash, known as erythema migrans, that appears at the site of the tick bite about a week after it occurred. Early symptoms of infection can include fever, headache, and tiredness. If untreated, the infection can progress to more severe neurological disorder manifestations such as loss of the ability to move one or both sides of the face, joint pain, severe headaches with neck stiffness, heart palpitations, tingling sensations, shooting pains, memory loss, and fatigue.

Coronavirus disease 2019, also known as COVID-19, is an infectious disease caused by the Severe Acute Respiratory Syndrome Corona Virus 2 (SARS-CoV-2). The disease was first identified in 2019 in Wuhan, Hubei province, China. Common symptoms of coronavirus infections include fever, cough, fatigue, shortness of breath, and loss of smell and taste. Even though the majority of cases result in mild symptoms and resolve within 2 weeks, some cases can progress to viral pneumonia, multi-organ failure, cytokine storm, and permanent tissue and organ damage, such as lung damage, heart and kidney damage, and death. The disease can be particularly serious with poor outcomes for those most at risk. Some of the more serious risk factors for severe COVID-19 illness include asthma, chronic lung disease, diabetes, serious heart conditions, chronic kidney disease being treated with dialysis, severe obesity, people aged 65 years and older, people in nursing homes or long-term care facilities, and those who are immunocompromised (such as patients undergoing cancer chemotherapy, immunologic treatments, or transplant recipients). However, there is increasing evidence of long term illness characterized by nervous system (CNS) involvement, lung/heart/renal impairment, and neurological manifestations in patients with prior COVID-19 infection. There is no direct correlation between the severity of the initial COVID-19 infection and subsequent long term sequelae. See, Ali A Asadi-Pooya and Leila Simani, Central nervous system manifestations of COVID-19: A systematic review, J Neurol Sci, 2020 Jun. 15; 413:116832. doi: 10.1016/j.jns.2020.116832. Epub 2020 Apr. 11. Many of these symptoms are associated with what is commonly known as “long COVID”, which is a condition characterized by long-term sequelae appearing or persisting after the typical convalescence period.

Antipurinergic agents constitute a family of compounds that antagonize purinergic receptors. These receptors are among the most abundant receptors in living organisms. They appeared early in evolution and are involved in regulating cellular functions. There are three known distinct classes of purinergic receptors, known as P1, P2X, and P2Y receptors. Also, purinergic signaling is a form of extracellular signaling. This signaling is mediated by purine nucleotides and nucleosides such as adenosine and adenosine triphosphate (ATP). This signaling involves the activation of purinergic receptors in the cell and/or in nearby cells, thereby regulating cellular functions. Purinergic receptors in the central nervous system play a crucial role in synaptic processes and mediating intercellular communications between neuron and glia cells, as a response to the release of adenosine triphosphate (ATP) or adenosine.

Chemical compounds that affect purinergic receptors are known. One of these is the compound, suramin, which was first synthesized in the early 1900s, and which has been found to have antipurinergic activity. Suramin is a medication used to treat the parasitic disease trypanosomiasis, which is caused by protozoa of the species Trypanosoma brucei and which is more commonly known as African sleeping sickness. The drug is also used to treat onchocerciasis, which is commonly known as river blindness. Because suramin has poor oral bioavailability, it is administered by injection into a vein. However, at the doses required for the treatment of African sleeping sickness (trypanosomiasis), suramin causes several side effects. These side effects include nausea, vomiting, diarrhea, abdominal pain, and a feeling of general discomfort. Other side effects include skin sensations such as crawling or tingling sensations, tenderness of the palms and soles, numbness of the extremities, watery eyes, rash, and photophobia. In addition, nephrotoxicity is common, as is peripheral neuropathy when the drug is administered at high doses. Regarding its pharmacokinetics, suramin is approximately 99-98% protein bound in the serum and has a half-life of 41-78 days, with an average of 50 days. Also, suramin is not extensively metabolized and is eliminated by the kidneys. Suramin is a large, polyanionic naphthylurea compound with six negative charges at physiological pH. Due to these factors, suramin cannot easily diffuse across biological membranes, which precludes it from crossing the blood-brain barrier or the blood-cerebrospinal fluid barrier. It is estimated that less than 1% of suramin crosses into the central nervous system. Therefore, there are many challenges with effectively utilizing suramin as an antipurinergic treatment.

From the foregoing it is apparent that the treatment of nervous system disorders remains challenging. Despite promising results from some early animal and human studies, it is recognized that much research is still needed to provide safe and effective means of administration for antipurinergic agents, such as suramin. It may be necessary or desirable to deliver appropriate levels of the drug to brain tissue while also minimizing systemic levels in the blood and other body tissues outside the CNS. However, it is difficult to deliver drugs across the blood-brain barrier (“BBB”), which is a natural protective mechanism of most mammals, including humans. The blood-brain barrier is a highly selective semipermeable border of endothelial cells that prevents solutes in the circulating blood from non-selectively crossing into the extracellular fluid of the central nervous system where neurons reside. Such delivery across the blood-brain barrier is even more challenging for higher molecular weight or highly charged compounds. For example, suramin has a molecular weight of approximately 1300 g/mol.

It has surprisingly been found in the present invention that the antipurinergic agent, suramin, can potentially be safely and effectively administered intranasally to achieve improvements in several behavioral deficits associated with disorders such as ASD, FXS, FXTAS, ME/CFS, PTSD, TS, PD, AS, and the CNS disorder manifestations associated with Lyme disease, COVID-19, and other viruses (e.g. Epstein Barr Human Herpesvirus 6 and 7, Herpes Simplex Virus, Cytomegalovirus, and others), including their long term effects. Specifically, it has been unexpectedly found that the methods of administering suramin employed herein demonstrated improvements in behavioral measures of anxiety or anxiety-like behavior, willingness to explore the environment, social interaction, spatial learning and memory, irritability, agitation and/or crying, lethargy and/or social withdrawal, stereotypic behavior, hyperactivity and/or noncompliance, and restrictive and/or repetitive behaviors. Furthermore, it has surprisingly been found in the present invention that the antipurinergic agent, suramin, can potentially be safely and effectively administered intranasally to achieve appropriate levels of the drug in brain tissue when certain penetration enhancers are employed. Specifically, it has surprisingly been found that penetration enhancers such as methyl Beta-cyclodextrin, caprylocaproyl macrogol-8 glycerides, and 2-(2-ethoxyethoxy)ethanol are particularly useful for preparing an intranasal suramin formulation having improved penetration of mucosal tissue. These compositions also have the further unexpected benefit of targeting brain tissue, while minimizing systemic blood levels of the suramin drug active. These compositions would therefore have utility for treating nervous system disorders and manifestations associated with them.

### SUMMARY OF THE INVENTION

Methods and compositions for the treatment of nervous system disorders such as cognitive, social, or behavioral disabilities are described. These disorders include neurodevelopmental disorders such as autism spectrum disorder (ASD), fragile X syndrome (FXS), fragile X-associated tremor/ataxia syndrome (FXTAS), myalgic encephalomyelitis/chronic fatigue syndrome (ME/CFS), post-traumatic stress syndrome (PTSD), Tourette's syndrome (TS), Parkinson's Disease (PD), Angelman syndrome (AS), and the CNS disorder manifestations often associated with Lyme disease and other tick-borne diseases, and the nervous system and central nervous system (CNS) disorders associated with COVID-19 and other viruses (e.g. Epstein Barr Human Herpesvirus 6 and 7, Herpes Simplex Virus, Cytomegalovirus, and others), including their long term effects. More specifically, the present invention provides methods and compositions for intranasal administration, i.e. delivery via a nasal route such as a nasal spray, comprising a therapeutically effective amount of the antipurinergic agent suramin, and pharmaceutically acceptable salts, esters, solvates, and prodrugs thereof. Examples of useful intranasal compositions comprise a therapeutically effective amount of suramin and a penetration aid for delivering therapeutically effective levels of the suramin active to the brain for treating the nervous system disorder, or symptoms, or behavioral manifestations thereof. These compositions are believed to minimize systemic levels of suramin while targeting higher levels in brain tissue, thereby helping to minimize potential drug toxicity and undesired side effects.

In some embodiments, the present invention provides a means to maximize delivery of suramin across the blood-brain barrier by intranasal administration to provide higher levels of a drug at the nasal mucosa. The present invention demonstrates that the transmucosal penetration of suramin, as determined in an in vitro assay, is significantly higher when delivered from a formulation comprising various penetration enhancers such as methyl Beta-cyclodextrin, caprylocaproyl macrogol-8 glycerides, and 2-(2-ethoxyethoxy)ethanol. The compositions of the present invention, when administered to mice, were found effective for delivering suramin to brain tissue and demonstrated brain tissue to plasma partitioning ratios. These compositions are designed to deliver the suramin active across the blood-brain barrier to brain tissue, while minimizing systemic levels to less than about a 3 micromolar plasma level and less than about 0.5 micromolar. The present invention is also based on the discovery that the intranasal administration of suramin in several animal models provides a benefit in delivering an improvement in study endpoints or behavioral manifestations associated with these nervous system disorders.

In other embodiments, the methods of the present invention can be achieved through intranasal administration of one or more doses of the suramin active ingredient. The dose or doses can be administered according to various treatment regimens.

In other embodiments, the present invention provides a device for patient administration or self-administration of the suramin active ingredient comprising a nasal spray inhaler containing an aerosol spray composition of the antipurinergic agent. This composition can comprise the suramin active ingredient and a pharmaceutically acceptable dispersant or solvent system, wherein the device is designed (or alternatively metered) to disperse an amount of the aerosol formulation by forming a spray that contains the dose of the suramin active ingredient. In other embodiments, the inhaler can comprise the suramin active ingredient as a fine powder, and further in combination with particulate dispersants and diluents, or alternatively with the suramin active ingredient combined to be incorporated within particles of the dispersant or to coat the particulate dispersants.

In other embodiments the present invention provides a method of treating a nervous system disorder such as a cognitive, social, or behavioral disability, or a neurodevelopmental disorder in a human patient in need thereof, comprising intranasally administering to said patient a pharmaceutical composition comprising an effective amount of suramin, or a pharmaceutically acceptable salt, ester, solvate, or prodrug thereof, wherein said composition provides an improvement in said patient in at least one of the following disorders, symptoms, or behavioral manifestations of the nervous system disorder selected from the group consisting of
a) anxiety or anxiety-like behavior,
b) willingness to explore the environment,
c) social interaction,
d) spatial learning and memory,
e) learning and memory,
f) irritability, agitation and or crying,
g) lethargy and/or social withdrawal,
h) stereotypic behavior,
i) hyperactivity and/or noncompliance, or
j) restrictive and/or repetitive behaviors.

In other embodiments, the present invention provides a method wherein said composition provides an improvement in said patient in at least one of the following disorders, symptoms, or behavioral manifestations of the nervous system disorder selected from the group consisting of
a) anxiety or anxiety-like behavior,
b) willingness to explore the environment,
c) social interaction,
d) spatial learning and memory, or
e) learning and memory.

In other embodiments, the present invention provides a method wherein the effective amount of suramin is a therapeutically effective amount.

In other embodiments, the present invention provides a wherein the pharmaceutically acceptable salt is selected from an alkali metal salt, an alkaline earth metal salt, and an ammonium salt.

In other embodiments, the present invention provides a method wherein said salt is a sodium salt.

In other embodiments, the present invention provides a method wherein said salt is the hexa-sodium salt.

In other embodiments the present invention provides a method wherein the nervous system disorder is selected from cognitive, social, or behavioral disabilities, and neurodevelopmental disorders.

In other embodiments, the present invention provides a method wherein the nervous system disorder is selected from the group consisting of autism spectrum disorder (ASD), fragile X syndrome (FXS), fragile X-associated tremor/ataxia syndrome (FXTAS), myalgic encephalomyelitis/chronic fatigue syndrome (ME/CFS), post-traumatic stress syndrome (PTSD), Tourette's syndrome (TS), Parkinson's Disease (PD), Angelman syndrome (AS), and the CNS disorder manifestations often associated with Lyme disease and other tick-borne diseases, and the nervous system and central nervous system (CNS) disorders associated with COVID-19 and other viruses (e.g. Epstein Barr Human Herpesvirus 6 and 7, Herpes Simplex Virus, Cytomegalovirus, and others), including their long term effects.

In other embodiments the present invention provides a method wherein the nervous system disorder is selected from autism spectrum disorder, FXS, or FXTAS.

In other embodiments the present invention provides a method wherein the nervous system disorder is autism spectrum disorder.

In other embodiments the present invention provides a method wherein said autism spectrum disorder is selected from the group consisting of autistic disorder, childhood disintegrative disorder, pervasive developmental disorder-not otherwise specified (PDD-NOS), and Asperger syndrome.

In other embodiments the present invention provides a method wherein said autism spectrum disorder manifests one or more symptoms or manifestations selected from difficulty communicating, difficulty interacting with others, and repetitive behaviors.

In other embodiments the present invention provides a method wherein the nervous system disorder is FXS.

In other embodiments the present invention provides a method wherein the nervous system disorder is FXTAS.

In other embodiments the present invention provides a method wherein the nervous system disorder is ME/CFS.

In other embodiments the present invention provides a method wherein the nervous system disorder is PTSD.

In other embodiments the present invention provides a method wherein the nervous system disorder is TS.

In other embodiments the present invention provides a method wherein the nervous system disorder is PD.

In other embodiments the present invention provides a method wherein the nervous system disorder is AS.

In other embodiments the present invention provides a method wherein the nervous system disorder is a central nervous system disorder manifestation associated with Lyme disease and other tick-borne diseases.

In other embodiments the present invention provides a method wherein the nervous system disorder is a nervous system or central nervous system (CNS) disorders associated with COVID-19 and other viruses (e.g. Epstein Barr Human Herpesvirus 6 and 7, Herpes Simplex Virus, Cytomegalovirus, and others), including their long term effects.

In other embodiments the present invention provides a method wherein the composition is administered or delivered, i.e. dosed, at least once daily, or at least twice daily, or at least once weekly, or at least twice weekly, or at least once biweekly (i.e. every two weeks), or at least once monthly, or at least once every 4 weeks.

In other embodiments the present invention provides a method wherein the composition is administered or delivered, i.e. dosed, at least once about every 41 days to about 78 days.

In other embodiments the present invention provides a method wherein the composition is administered or delivered, i.e. dosed, at least once about every 50 days.

In other embodiments the present invention provides a method wherein the composition is administered or delivered, i.e. dosed, at least once per a time interval based on the average half-life of suramin.

In other embodiments the present invention provides a method wherein the composition exhibits, i.e. is capable of providing, a penetration rate of about 1 micrograms/cm² per hour to about 200 micrograms/cm² per hour of suramin, based on the suramin active, through cultured human airway tissue.

In other embodiments the present invention provides a method wherein the plasma level of the suramin in the patient is maintained at less than about 3 micromolar (μM), or less than about 2.75 micromolar, or less than about 2.5 micromolar, or less than about 2 micromolar, or less than about 1 micromolar, or less than about 0.5 micromolar based on the suramin active.

In other embodiments the present invention provides a method wherein the brain tissue level of the suramin in the patient is from about 1 ng/ml to about 1000 ng/ml.

In other embodiments the present invention provides a method wherein the brain tissue level of the suramin in the patient is at least about 1 ng/ml, or at least about 10 ng/ml, or at least about 50 ng/ml, or at least about 100 ng/ml, or at least about 250 ng/ml, or at least about 500 ng/ml.

In other embodiments the present invention provides a method wherein the brain tissue to blood plasma partitioning ratio for the suramin is at least about 0.05, or at least about 0.1, or at least about 0.25, or at least about 0.50.

In other embodiments the present invention provides a method wherein the AUC for the plasma level for the suramin active for the patient is less than about 80 μg*day/L or is less than about 75 μg*day/L, or is less than about 50 μg*day/L, or is less than about 25 μg*day/L, or is less than about 10 μg*day/L.

In other embodiments the present invention provides a method wherein the Cₘₐₓ for the plasma level for the suramin active for the patient is less than about 75 micromolar, or is less than about 7.5 micromolar, or is less than about 0.1 micromolar, and optionally at least about 0.01 micromolar, based on a single dose.

In other embodiments the present invention provides a method wherein treating said autism spectrum disorder, FXS, or FXTAS comprises improving one or more symptoms or manifestations of said patient relative to symptoms or manifestations of said patient prior to said administration, wherein said one or more symptoms or manifestations are selected from difficulty communicating, difficulty interacting with others, and repetitive behaviors.

In other embodiments the present invention provides a method wherein treating said autism spectrum disorder, FXS, or FXTAS comprises improving an assessment score of said patient relative to a score from said patient prior to said administration.

In other embodiments, the present invention provides a method wherein said assessment score of said patient is improved by 10% or more relative to a score from said patient prior to said administration.

In other embodiments the present invention provides a method wherein the assessment score is selected from ABC, ADOS, ATEC, CARS CGI, and SRS. These assessment score acronyms are defined, below, in the definitions section.

In other embodiments the present invention provides a method wherein the composition is a nasal spray.

In other embodiments the present invention provides a method wherein the composition is an aqueous composition.

In other embodiments the present invention provides a method wherein the composition is a powdered composition.

In other embodiments the present invention provides a method wherein the composition is a mucoadhesive sprayable fluid gel.

In other embodiments the present invention provides a method of treating a nervous system disorder such as a cognitive, social, or behavioral disability, or a neurodevelopmental disorder in a human patient in need thereof, comprising intranasally administering to said patient a pharmaceutical composition comprising an effective amount of suramin, or a pharmaceutically acceptable salt, ester, solvate, or prodrug thereof, wherein said composition, when evaluated in an animal model, provides an improvement in at least one of the following behavioral manifestations selected from the group consisting of:
a) light/dark test (LDT),
b) locomotor activity test,
c) social interaction test,
d) Morris Water Maze Test (MWM), or
e) step through passive avoidance test.

In other embodiments there present invention provides a method wherein said animal model is a transgenic FMR-1 mouse model.

In other embodiments the present invention provides a use of suramin, or a pharmaceutically acceptable salt, ester, solvate, or prodrug thereof in the manufacture of a medicament for intranasal delivery of an effective amount of suramin for treating a nervous system disorder such as a cognitive, social, or behavioral disability, or a neurodevelopmental disorder in a human patient in need thereof, wherein said composition provides an improvement in said patient in at least one of the following disorders, symptoms, or behavioral manifestations of the nervous disorder selected from the group consisting of
a) anxiety or anxiety-like behavior,
b) willingness to explore the environment,
c) social interaction,
d) spatial learning and memory,
e) learning and memory,
f) irritability, agitation and or crying,
g) lethargy and/or social withdrawal,
h) stereotypic behavior,
i) hyperactivity and/or noncompliance, or
j) restrictive and/or repetitive behaviors.

In other embodiments the present invention provides a device for performing the methods of the present invention, comprising a nasal spray inhaler for intranasally administering said pharmaceutical composition.

In other embodiments, the present invention provides methods and compositions wherein the amount of suramin is based on the suramin active ingredient (i.e. the chemical entity), using a molecular weight (i.e. a molar mass) of 1297.26 grams/mole, or approximately 1300 grams per/mole.

In other embodiments, the present invention provides a method wherein the composition comprises from about 0.01 mg to about 200 mg per unit dosage of suramin, based on the suramin active.

In other embodiments, the present invention provides a method wherein the composition comprises from about 0.01 mg to about 100 mg per unit dosage of suramin, based on the suramin active.

In other embodiments, the present invention provides a method wherein the composition comprises from about 0.01 mg to about 50 mg per unit dosage of suramin, based on the suramin active.

In other embodiments, the present invention provides a method wherein the composition comprises from about 0.01 mg to about 25 mg per unit dosage of suramin, based on the suramin active.

In other embodiments, the present invention provides a method wherein the composition comprises from about 0.01 mg to about 10 mg per unit dosage of suramin, based on the suramin active.

In other embodiments, the present invention provides a method wherein the composition comprises from about 0.1 mg/kg per week to about 20 mg/kg per week of suramin, based on the suramin active and the weight of the patient.

In other embodiments, the present invention provides a method wherein the composition comprises from about 0.025 mg/kg to about 10 mg/kg per unit dosage of suramin, based on the suramin active and the weight of the patient.

In other embodiments, the present invention provides a method wherein the composition comprises from about 0.05 mg/kg to about 6 mg/kg per unit dosage of suramin, based on the suramin active and the weight of the patient.

In other embodiments, the present invention provides a method wherein the composition comprises from about 0.0476 mg/kg to about 5.720 mg/kg of the per unit dosage of suramin, based on the suramin active and the weight (mass) of the patient.

In other embodiments, the present invention provides a method wherein the composition comprises less than about 1 mg/kg per unit dosage of suramin, based on the suramin active and the weight of the patient.

In other embodiments, the present invention provides a method wherein the composition comprises less than about 0.5 mg/kg per unit dosage of suramin, based on the suramin active and the weight of the patient.

In other embodiments, the present invention provides a method wherein the composition comprises less than about 0.25 mg/kg per unit dosage of suramin, based on the suramin active and the weight of the patient.

In other embodiments, the present invention provides a method wherein the composition comprises less than about 0.1 mg/kg per unit dosage of suramin, based on the suramin active and the weight of the patient.

In other embodiments, the present invention provides a method wherein the composition comprises less than about 400 mg/m 2 per unit dosage of suramin, based on the suramin active and the body surface area (BSA) of the patient.

In other embodiments, the present invention provides a method wherein the composition comprises less than about 200 mg/m 2 per unit dosage of suramin, based on the suramin active and the body surface area (BSA) of the patient.

In other embodiments, the present invention provides a method wherein the composition comprises less than about 100 mg/m 2 per unit dosage of suramin, based on the suramin active and the body surface area (BSA) of the patient.

In other embodiments, the present invention provides a method wherein the composition comprises less than about 50 mg/m 2 per unit dosage of suramin, based on the suramin active and the body surface area (BSA) of the patient.

In other embodiments, the present invention provides a method wherein the composition comprises less than about 25 mg/m 2 per unit dosage of suramin, based on the suramin active and the body surface area (BSA) of the patient.

In other embodiments, the present invention provides a method wherein the composition comprises from about 10 mg/m 2 to about 300 mg/m 2 per unit dosage of suramin, based on the suramin active and the body surface area (BSA) of the patient.

In other embodiments, the present invention provides a method wherein the AUC for the plasma level for the suramin active for the patient is less than about 80 μg*day/L.

In other embodiments, the present invention provides a method wherein the AUC for the plasma level for the suramin active for the patient is less than about 75 μg*day/L.

In other embodiments, the present invention provides a method wherein the AUC for the plasma level for the suramin active for the patient is less than about 50 μg*day/L.

In other embodiments, the present invention provides a method wherein the AUC for the plasma level for the suramin active for the patient is less than about 25 μg*day/L.

In other embodiments, the present invention provides a method wherein the AUC for the plasma level for the suramin active for the patient is less than about 10 μg*day/L.

In other embodiments, the present invention provides a method wherein the Cₘₐₓ for the plasma level for the suramin active for the patient is less than about 75 micromolar, per dose of drug composition.

In other embodiments, the present invention provides a method wherein the Cₘₐₓ for the plasma level for the suramin active for the patient is less than about 7.5 micromolar, per dose of drug composition.

In other embodiments, the present invention provides a method wherein the Cₘₐₓ for the plasma level for the suramin active for the patient is less than about 0.1 micromolar. Although there is no minimum Cₘₐₓ the amount can generally be above about 0.01 micromolar per dose of drug composition.

In other embodiments, the present invention provides a method wherein each unit dosage comprises about 0.01 ml to about 0.5 ml of liquid.

In other embodiments, the present invention provides a method wherein each unit dosage comprises about 0.1 ml of liquid.

In other embodiments, the present invention provides a method wherein the composition exhibits, i.e. is capable of providing, a penetration rate of about 1 micrograms/cm² per hour to about 200 micrograms/cm² per hour of suramin, based on the suramin active, through cultured human airway tissue.

In other embodiments, the present invention provides a method wherein the composition further comprises an agent selected for osmolality control.

In other embodiments, the present invention provides a method wherein the composition further comprises an agent selected for osmolality control, wherein said agent is selected from a salt, such as for example sodium chloride.

In other embodiments, the present invention provides a method wherein the compositions further comprise a thickening agent.

In other embodiments, the present invention provides a method wherein said autism spectrum disorder includes one or more symptoms selected from difficulty communicating, difficulty interacting with others, and repetitive behaviors.

In other embodiments, the present invention provides a method wherein treating said ASD, FXS, FXTAS, ME/CFS, PTSD, TS, PD, AS, or the CNS disorder manifestations associated with Lyme disease, COVID-19, other viruses (e.g. Epstein Barr Human Herpesvirus 6 or 7, Herpes Simplex Virus, Cytomegalovirus, and others), including their long term effects comprises improving one or more symptoms relative to symptoms of said patient prior to said administration, wherein said one or more symptoms are selected from difficulty communicating, difficulty interacting with others, and repetitive behaviors.

In other embodiments, the present invention provides a method wherein treating said ASD, FXS, FXTAS, ME/CFS, PTSD, TS, PD, AS, or the CNS disorder manifestations associated with Lyme disease, COVID-19, other viruses (e.g. Epstein Barr Human Herpesvirus 6 or 7, Herpes Simplex Virus, Cytomegalovirus, and others), including their long term effects comprises improving an assessment score of said patient relative to a score from said patient prior to said administration.

In other embodiments, the present invention provides a method wherein an assessment score of said patient is improved by 10% or more relative to a score from said patient prior to said administration.

In other embodiments, the present invention provides a method wherein the assessment score is selected from ABC, ADOS, ATEC, CARS CGI, and SRS.

In other embodiments, the present invention provides a method wherein an ADOS score of the patient is improved by 1.6 or more relative to a score prior to said administration, or a corresponding performance improvement on a similar test.

In other embodiments, the present invention provides a method wherein the p-value of improvement of said ADOS score or similar test is 0.05 or less.

In other embodiments, the present invention provides a method wherein the size effect of improvement of said ADOS score or similar test is about 1 or more.

In other embodiments, the present invention provides a method wherein the size effect of improvement of said ADOS score or similar test is about 2.9 or more.

In other embodiments, the present invention provides an intranasal delivery pharmaceutical composition for treating a nervous system disorder comprising:
(a) therapeutically effective amount of suramin, or a pharmaceutically acceptable salt, ester, solvate, or prodrug thereof, and
(b) a penetration enhancer.

In other embodiments, the present invention provides a composition further comprising (c) water.

In other embodiments, the present invention provides a device for patient administration, including administration selected from self-administration and administration to the patient by an individual other than the patient, comprising a nasal spray inhaler for administering a composition comprising suramin, or a pharmaceutically acceptable salt, ester, solvate, or prodrug thereof, wherein the device is designed (or alternatively metered) to disperse an amount of the suramin for treating a nervous system disorder in a patient in need thereof.

In other embodiments, the present invention provides a device wherein the antipurinergic agent comprises a composition selected from a solution, an emulsion, or a powder.

These and other embodiments of the present invention will become apparent from the disclosure herein.

### BRIEF DESCRIPTION OF THE DRAWINGS

FIG. 1 shows a plot of cumulative drug permeation, in mg, versus time, in hours, for aqueous suramin compositions with three different penetration enhancers versus a control composition with no penetration enhancer.

FIG. 2 shows a plot of cumulative drug permeation, in mg, versus time, in hours, for aqueous suramin compositions with five different penetration enhancers versus a control composition with no penetration enhancer.

FIG. 3 shows a plot of the total concentration, in ng/ml, of suramin in plasma versus brain tissue in mice when administered by intraperitoneal (IP) injection, 20 mg/kg, weekly to the mice beginning at 9 weeks of age and continuing for four weeks (i.e. given at age weeks 9, 10, 11 and 12).

FIG. 4 shows a plot comparing the total concentration, in ng/ml, of suramin in plasma versus brain tissue in mice when administered intranasally (IN) daily for 28 days. A composition of the present invention comprising IN suramin, at a concentration of 100 mg/mL×6 mL per spray, was administered as one spray per nostril, one time per day, (interval of each application is around 2 minutes to ensure absorption) for 28 days (total of 56 sprays over 28 day period) beginning at 9 weeks of age (i.e. given daily during age weeks 9, 10, 11 and 12).

FIG. 5 shows a plot comparing the total concentration, in ng/ml, of suramin in plasma versus brain tissue in mice when administered intranasally (IN) every other day for 28 days. A composition of the present invention comprising IN suramin, at a concentration of 100 mg/mL×6 mL per spray, was administered as one spray per nostril, every other day, (interval of each application is around 2 minutes to ensure absorption) for 28 days (total of 28 sprays over 28 day period) beginning at 9 weeks of age (i.e. given daily during age weeks 9, 10, 11 and 12).

FIG. 6 shows a plot comparing the total concentration, in ng/ml, of suramin in plasma versus brain tissue in mice when administered intranasally (IN) once per week for 4 weeks. A composition of the present invention comprising IN suramin, at a concentration of 100 mg/mL×6 mL per spray, was administered as one spray per nostril, one time per week, (interval of each application is around 2 minutes to ensure absorption) for 4 weeks (28 days) (total of 8 sprays over 28 day period) beginning at 9 weeks of age (i.e. given daily during age weeks 9, 10, 11 and 12).

FIG. 7 shows a plot comparing the total percentage of suramin in plasma in mice when administered by intraperitoneal (IP) injection once weekly for 4 weeks (28 days), intranasally (IN) daily for 28 days, intranasally (IN) every other day for 28 days, and intranasally (IN) once per week for 4 weeks (28 days).

FIG. 8 shows a plot comparing the total percentage of suramin in brain tissue in mice when administered by intraperitoneal (IP) injection once weekly for 4 weeks (28 days), intranasally (IN) daily for 28 days, intranasally (IN) every other day for 28 days, and intranasally (IN) once per week for 4 weeks (28 days).

FIG. 9 shows a plot comparing the total percentage of suramin in plasma versus brain tissue in mice when administered by intraperitoneal (IP) injection once weekly for 4 weeks (28 days), intranasally (IN) daily for 28 days, intranasally (IN) every other day for 28 days, and intranasally (IN) once per week for 4 weeks (28 days).

FIG. 10 shows a plot comparing the brain tissue to plasma partitioning ratio of suramin in mice when administered by intraperitoneal (IP) injection once weekly for 4 weeks (28 days), intranasally (IN) daily for 28 days, intranasally (IN) every other day for 28 days, and intranasally (IN) once per week for 4 weeks (28 days).

FIG. 11 shows a plot comparing time to entry of the dark zone for a light/dark preference test in mice when treated with suramin when administered by intraperitoneal (IP) injection once weekly for 4 weeks (28 days), intranasally (IN) daily for 28 days, intranasally (IN) every other day for 28 days, and intranasally (IN) once per week for 4 weeks (28 days). Also, shown is data for saline and wild type controls.

FIGS. 12A and 12B show plots of the time spent in the light zone for a light/dark preference test in mice when treated with suramin when administered by intraperitoneal (IP) injection once weekly for 4 weeks (28 days), intranasally (IN) daily for 28 days, intranasally (IN) every other day for 28 days, and intranasally (IN) once per week for 4 weeks (28 days). FIG. 12A shows the time measured in minutes. FIG. 12B shows the time expressed as a percentage. Also, shown is data for saline and wild type controls.

FIG. 13 shows a plot of the number of light zone entries for a light/dark preference test in mice when treated with suramin when administered by intraperitoneal (IP) injection once weekly for 4 weeks (28 days), intranasally (IN) daily for 28 days, intranasally (IN) every other day for 28 days, and intranasally (IN) once per week for 4 weeks (28 days). Also, shown is data for saline and wild type controls.

FIG. 14 shows a plot of the active time in minutes per hour for a locomotor activity test in mice when treated with suramin when administered by intraperitoneal (IP) injection once weekly for 4 weeks (28 days), intranasally (IN) daily for 28 days, intranasally (IN) every other day for 28 days, and intranasally (IN) once per week for 4 weeks (28 days). The grey areas of the plot show the period when the animals are in a simulated “dark” or night period. Also, shown is data for saline and wild type controls.

FIG. 15 shows a plot of the travel distance in centimeters per hour for a locomotor activity test in mice when treated with suramin when administered by intraperitoneal (IP) injection once weekly for 4 weeks (28 days), intranasally (IN) daily for 28 days, intranasally (IN) every other day for 28 days, and intranasally (IN) once per week for 4 weeks (28 days). The grey areas of the plot show the period when the animals are in a simulated “dark” or night period. Also, shown is data for saline and wild type controls.

FIG. 16 shows a plot of the rearing count (standing on rear limbs) per hour for a locomotor activity test in mice when treated with suramin when administered by intraperitoneal (IP) injection once weekly for 4 weeks (28 days), intranasally (IN) daily for 28 days, intranasally (IN) every other day for 28 days, and intranasally (IN) once per week for 4 weeks (28 days). The grey areas of the plot show the period when the animals are in a simulated “dark” or night period. Also, shown is data for saline and wild type controls.

FIG. 17 shows a plot of the habituation for 0 to 5 minutes and the occupancy time for 0 to 5 minutes for a social interaction study in mice when treated with suramin when administered by intraperitoneal (IP) injection once weekly for 4 weeks (28 days), intranasally (IN) daily for 28 days, intranasally (IN) every other day for 28 days, and intranasally (IN) once per week for 4 weeks (28 days). Also, shown is data for saline and wild type controls. Bar graphs left to right are: IP Suramin, IP Saline, IN Suramin—Daily, IN Suramin—Every 2 Days, IN Suramin—Weekly, and WT—C57BL/6+Saline.

FIG. 18 shows a plot of the sociability analysis (0 to 5 minutes) depicting occupancy time in minutes for stranger compartments 1 and 2 for a social interaction study in mice when treated with suramin when administered by intraperitoneal (IP) injection once weekly for 4 weeks (28 days), intranasally (IN) daily for 28 days, intranasally (IN) every other day for 28 days, and intranasally (IN) once per week for 4 weeks (28 days). Also, shown is data for saline and wild type controls. Bar graphs left to right are: IP Suramin, IP Saline, IN Suramin—Daily, IN Suramin—Every 2 Days, IN Suramin—Weekly, and WT—C57BL/6+Saline.

FIG. 19 shows a plot of social novelty with occupancy time in minutes measured in each compartment after the introduction of a new mouse for a social interaction study in mice when treated with suramin when administered by intraperitoneal (IP) injection once weekly for 4 weeks (28 days), intranasally (IN) daily for 28 days, intranasally (IN) every other day for 28 days, and intranasally (IN) once per week for 4 weeks (28 days). Also, shown is data for saline and wild type controls. Bar graphs left to right are: IP Suramin, IP Saline, IN Suramin—Daily, IN Suramin—Every 2 Days, IN Suramin—Weekly, and WT—C57BL/6+Saline.

FIG. 20 shows a plot of the acquisition test escape latency in seconds in the Morris Water Maze Test in mice when treated with suramin when administered by intraperitoneal (IP) injection once weekly for 4 weeks (28 days), intranasally (IN) daily for 28 days, intranasally (IN) every other day for 28 days, and intranasally (IN) once per week for 4 weeks (28 days). Also, shown is data for saline and wild type controls. Graph lines top to bottom at first entries of graph (Day 1): IP Saline, WT, IN Suramin—Every 2 Days, IP Suramin, IN Suramin—Weekly, and IN Suramin—Daily.

FIG. 21 shows a plot of the probe test in seconds to locate the escape platform in the Morris Water Maze Test in mice when treated with suramin when administered by intraperitoneal (IP) injection once weekly for 4 weeks (28 days), intranasally (IN) daily for 28 days, intranasally (IN) every other day for 28 days, and intranasally (IN) once per week for 4 weeks (28 days). Also, shown is data for saline and wild type controls.

FIG. 22 shows a plot of dark zone latency in seconds for the training day and the test day 24 hours later testing learning and memory in mice in a step through passive avoidance test evaluating suramin when administered by intraperitoneal (IP) injection once weekly for 4 weeks (28 days), intranasally (IN) daily for 28 days, intranasally (IN) every other day for 28 days, and intranasally (IN) once per week for 4 weeks (28 days). Also, shown is data for saline and wild type controls. Bar graphs left to right are: IP Suramin, IP Saline, IN Suramin—Daily, IN Suramin—Every 2 Days, IN Suramin—Weekly, and WT—C57BL/6+Saline.

FIGS. 23A and 23B show plots of the time spent in the light zone in a step through passive avoidance test in mice evaluating suramin when administered by intraperitoneal (IP) injection once weekly for 4 weeks (28 days), intranasally (IN) daily for 28 days, intranasally (IN) every other day for 28 days, and intranasally (IN) once per week for 4 weeks (28 days). FIG. 23A shows the time measured in minutes. FIG. 23B shows the time expressed as a percentage. Also, shown is data for saline and wild type controls.

FIG. 24 shows a plot of the number of dark zone entries in a step through passive avoidance test in mice evaluating suramin when administered by intraperitoneal (IP) injection once weekly for 4 weeks (28 days), intranasally (IN) daily for 28 days, intranasally (IN) every other day for 28 days, and intranasally (IN) once per week for 4 weeks (28 days). Also, shown is data for saline and wild type controls.

FIG. 25 shows a plot of the number of light zone entries in a step through passive avoidance test in mice evaluating suramin when administered by intraperitoneal (IP) injection once weekly for 4 weeks (28 days), intranasally (IN) daily for 28 days, intranasally (IN) every other day for 28 days, and intranasally (IN) once per week for 4 weeks (28 days). Also, shown is data for saline and wild type controls.

### DETAILED DESCRIPTION OF THE INVENTION

### Definitions

As used herein, the following terms and abbreviations have the indicated meanings unless expressly stated to the contrary.

The term “ABC”, as used herein is also known as the “Aberrant Behavior Checklist” and is a rating scale for evaluating autism.

The term “ADOS”, as used herein is also known as “The Autism Diagnostic Observation Schedule” is an instrument for diagnosing and assessing autism. The protocol consists of a series of structured and semi-structured tasks that involve social interaction between the examiner and the person under assessment.

The term “AS”, as used herein is also known as Angelman syndrome.

The term “ASD”, as used herein is also known as Autism Spectrum Disorder.

The term “ATEC”, as used herein is also known as “The Autism Treatment Evaluation Checklist”, is a 77-item diagnostic assessment tool that was developed at the Autism Research Institute. The ATEC was originally designed to evaluate the effectiveness of autism treatments but is also used as a screening tool.

The term “AUC”, also known as “Area Under the Curve” as used herein is standard terminology in pharmacology, specifically pharmacokinetics. The term refers to the definite integral of a curve that describes the variation of a drug concentration in blood plasma as a function of time. In practice, the drug concentration is measured at certain discrete points in time and the trapezoidal rule is used to estimate AUC. The AUC gives a measure of bioavailability and refers to the fraction of drug absorbed systemically. Knowing this, one can also determine the clearance for the drug. The AUC reflects the actual body exposure to drug after administration of a dose of the drug and is usually expressed in mg*h/L or μg*h/L (where “h” stands for hours). Alternatively, the AUC can be expressed in mg*day/L or μg*day/L. Note that the asterisk, “*”, in the units for AUC denotes a multiplication and that in alternative notations a dot “·” or multiplication symbol “x” is used.

The term “based on the suramin active” as used herein is meant to provide a basis for determining or calculating the amount of suramin based on the suramin molecular weight (i.e. a molar mass) of 1297.26 grams/mole. This is an important consideration for determining the amount of suramin when it is delivered as a salt or other form, having a different total molecular weight, such as for example the hexa-sodium salt which would have a molecular weight (i.e. a molar mass) of 1429.15 grams/mole.

The term “CARS”, as used herein is also known as “The Childhood Autism Rating Scale” and is a behavior rating scale intended to help diagnose and evaluate autism.

The term “CGI”, as used herein is also known as “The Clinical Global Impression” rating scale and is a measure of symptom severity, treatment response and the efficacy of treatments in treatment studies of patients with psychological disorders.

The term “Cₘₐₓ” as used herein is standard terminology in pharmacology, specifically pharmacokinetics, for defining the maximum (or peak) serum concentration that a drug achieves in a specified compartment or test area of the body after the drug has been administered and before the administration of a second dose.

The term “FXS” as used herein means fragile X syndrome.

The term “FXTAS” as used herein means fragile X-associated tremor/ataxia syndrome.

The term “IN” as used herein means intranasal.

The term “Long COVID Syndrome”, as used herein means persisting symptoms after COVID-19 infection which last beyond about 12 weeks from the initial infection.

The term “ME/CFS”, as used herein is also known as myalgic encephalomyelitis/chronic fatigue syndrome (ME/CFS).

The term “nasal spray” as used herein means a product that is intended to be delivered from a spray or aerosolizing device, which can for example be in the form of a liquid, powder, gel, foam, cream, ointment, or other sprayable composition.

The term “PD”, as used herein is also known as Parkinson's Disease.

The term “pharmaceutically acceptable” is used herein with respect to the compositions, in other words the formulations, of the present invention, and also with respect to the pharmaceutically acceptable salts, esters, solvates, and prodrugs of suramin. The pharmaceutical compositions of the present invention comprise a therapeutically effective amount of suramin and a pharmaceutically acceptable carrier. These carriers can contain a wide range of excipients. Pharmaceutically acceptable carriers are those conventionally known carriers having acceptable safety profiles. The compositions are made using common formulation techniques. See, for example, Remington's Pharmaceutical Sciences, 17th edition, edited by Alfonso R. Gennaro, Mack Publishing Company, Easton, PA, 17th edition, 1985. Regarding pharmaceutically acceptable salts, these are described below.

The term “PTSD”, as used herein is also known as “Post-Traumatic Stress Disorder or Syndrome”.

The term “SRS”, as used herein is also known as the “Social Responsiveness Scale” which is used herein is a measure of autism spectrum disorder.

The term “subject” means a human patient or animal in need of treatment or intervention for a nervous system disorder.

The term “therapeutically effective” means an amount of suramin needed to provide a meaningful or demonstrable benefit, as understood by medical practitioners, to a subject, such as a human patient in need of treatment. Conditions, intended to be treated include, for example, autistic disorder, childhood disintegrative disorder, pervasive developmental disorder-not otherwise specified (PDD-NOS), and Asperger syndrome. For example, a meaningful or demonstrable benefit can be assessed or quantified using various clinical parameters. The demonstration of a benefit can also include those provided by models, including but not limited to in vitro models, in vivo models, and animal models. An example of such an in vitro model is the permeation of the drug active studied using cultured human airway tissues (EpiAirway AIR-100) to simulate permeation across the nasal mucosal membrane.

The term “TS”, as used herein is also known as “Tourette's syndrome”.

The term “intranasal” (“IN”) as used herein with respect to the pharmaceutical compositions and actives therein, means a composition that is administered to the nose or by way of the nose for delivery across the mucosal membrane inside the nasal cavity. This membrane is a well vascularized thin mucosa. Furthermore, this mucosa is in close proximity to the brain and provides a means to maximize the transport of drugs across the blood-brain barrier, in some cases via different nerves and along their nerve sheaths, including the olfactory and trigeminal nerves. The blood-brain barrier is a highly selective semipermeable border that separates the circulating blood from the brain and extracellular fluid in the central nervous system. Delivering therapeutic agents to specific regions of the brain presents a challenge to treatment of many brain disorders. It should be noted that transmucosal administration is different from topical administration and transdermal administration. The U.S. Food & Drug Administration has provided a standard for a wide range of routes of administration for drugs, i.e. “Route of Administration”. The following definitions are provided by the FDA for example for endosinusial, intracerebral, intranasal, nasal, topical, transdermal, and transmucosal routes of drug administration. The routes of administration useful in the present invention include endosinusial, intranasal, and nasal, recognizing that transmucosal delivery through the nasal mucosa is also intended. These routes of administration are distinguished from inhalation which is intended to deliver a drug into the lungs and bronchi. See for example, U.S. Pat. No. 8,785,500 to Charney et al., issued Jul. 22, 2014, which discloses examples of methods and compositions for intranasally administering a drug active.

| | | | | NCI* |
|---|---|---|---|---|
| | | | | CON- |
| | | SHORT | FDA | CEPT |
| NAME | DEFINITION | NAME | CODE | ID |
| ENDO- | Administration within the | E-SINUS | 133 | C38206 |
| SINUSIAL | nasal sinuses of the | | | |
| | head. | | | |
| INTRA- | Administration within the | I-CERE | 404 | C38232 |
| CEREBRAL | cerebrum. | | | |
| INTRASINAL | Administration within the | I-SINAL | 010 | C38262 |
| | nasal or periorbital | | | |
| | sinuses. | | | |
| NASAL | Administration to the | NASAL | 014 | C38284 |
| | nose; administered by | | | |
| | way of the nose. | | | |
| TOPICAL | Administration to a | TOPIC | 011 | C38304 |
| | particular spot on the | | | |
| | outer surface of the body. | | | |
| TRANS- | Administration through | T- | 358 | C38305 |
| DERMAL | the dermal layer of the | DERMAL | | |
| | skin to the systemic | | | |
| | circulation by diffusion. | | | |
| TRANS- | Administration across the | T- | 122 | C38283 |
| MUCOSAL | mucosa. | MUCOS | | |

*National Cancer Institute

See, www.fda.gov/Drugs/DevelopmentApprovalProcess/FormsSubmissionRequirements/Ele ctronicSubmissions/DataStandardsManualmonographs/ucm071667.htm.

The terms “treat,” “treating” or “treatment,” as used herein, include alleviating, abating or ameliorating the condition, e.g. autism and other nervous system disorders, or preventing or reducing the risk of contracting the condition or exhibiting the symptoms of the condition, ameliorating or preventing the underlying causes of the symptoms, inhibiting the condition, arresting the development of the condition, relieving the condition, causing regression of the condition, or stopping the symptoms of the condition, either prophylactically and/or therapeutically.

The abbreviation “WT” means wild-type, which is a phenotype, genotype, or gene that predominates in a natural population of in contrast to that of mutant forms.

The methods of treatment using suramin or a pharmaceutically acceptable salt, ester, solvate, or prodrug thereof or the pharmaceutical compositions of the present invention, in various embodiments also include the use of suramin or a pharmaceutically acceptable salt, ester, solvate, or prodrug thereof in the manufacture of a medicament for the desired treatment, such as for a nervous system disorder.

Suramin

The present invention utilizes a therapeutically effective amount of the antipurinergic agent suramin, or a pharmaceutically acceptable salt, ester, solvate, or prodrug thereof for treating a nervous system disorder. Some embodiments also include a penetration enhancer, and also a pharmaceutically acceptable carrier for providing intranasal administration.

Suramin is a sulfonic acid drug compound, corresponding to the CAS Registry Number 145-63-1 and ChemSpider ID 5168. One of the chemical names for suramin is: 1,3,5-Naphthalenetrisulfonic acid, 8,8′-[carbonylbis[imino-3,1-phenylenecarbonylimino(4-methyl-3,1-phenylene)carbonylimino]]bis-. The compound is a medication used to treat African sleeping sickness (trypanosomiasis) and river blindness (onchocerciasis) and is known by the trade names Antrypol, 309 F, 309 Fourneau, Bayer 205, Germanin, Moranyl, Naganin, and Naganine. However, the drug is not approved by the US FDA. The drug is administered by venous injection. Suramin has been reported to have been studied in a mouse model of autism and in a Phase I/II human trial. See, Naviaux, J. C. et al., “Reversal of autism-like behaviors and metabolism in adult mice with single-dose antipurinergic therapy”. Translational Psychiatry. 4 (6): e400 (2014). Also, see, Naviaux, R. K. et al., “Low-dose suramin in autism spectrum disorder: a small, phase I/II, randomized clinical trial”, Annals of Clinical and Translational Neurology, 2017 May 26:4(7):491-505.

Suramin is reported to have a half-life of between about 41 to 78 days with an average of 50 days. See, Phillips, Margaret A.; Stanley, Jr, Samuel L. (2011). “Chapter 50: Chemotherapy of Protozoal Infections: Amebiasis, Giardiasis, Trichomoniasis, Trypanosomiasis, Leishmaniasis, and Other Protozoal Infections”. In Brunton, Laurence L. Chabner, Bruce A.; Knollmann, Bjorn Christian (eds.). Goodman and Gilman's The Pharmacological Basis of Therapeutics (12th ed.). McGraw Hill. pp. 1437-1438.

The chemical formula of suramin is C₅₁H₄₀N₆O₂₃S₆. Suramin therefore has a molecular weight (i.e. a molar mass) of 1297.26 grams/mole. Suramin is usually delivered as a sodium sulfonate salt, such as the hexa-sodium salt, which has a molecular weight (i.e. a molar mass) of 1429.15 grams/mole. Note that these molecular weight values will vary slightly depending on what atomic weight values are used for the calculations. The chemical structure for suramin is shown immediately below.

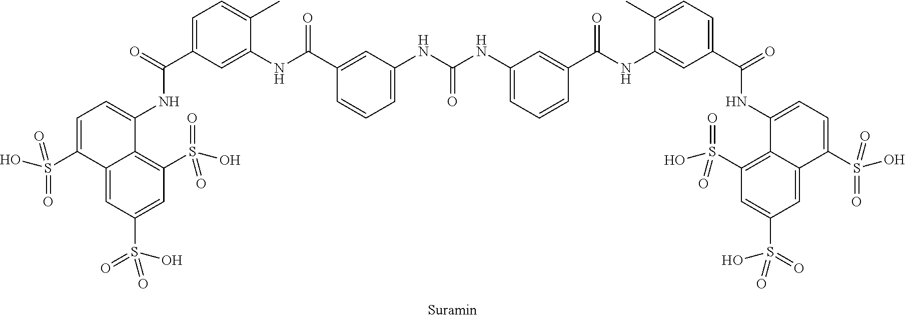 [SMILES: Cc1ccc(C(=O)Nc2ccc(S(=O)(=O)O)c3cc(S(=O)(=O)O)cc(S(=O)(=O)O)c23)cc1NC(=O)c1cccc(NC(=O)Nc2cccc(C(=O)Nc3cc(C(=O)Nc4ccc(S(=O)(=O)O)c5cc(S(=O)(=O)O)cc(S(=O)(=O)O)c45)ccc3C)c2)c1]

Pharmaceutically acceptable salts, esters, solvates, and prodrugs of suramin are useful for the methods and compositions of the present invention. As used herein, “pharmaceutically acceptable salts, esters, solvates, and prodrugs” refer to derivatives of suramin. Examples of pharmaceutically acceptable salts include, but are not limited to, alkali metal salts, alkaline earth metal salts, and ammonium salts. Examples of alkali metal salts include lithium, sodium, and potassium salts. Examples of alkaline earth metal salts include calcium and magnesium salts. The ammonium salt, NH4+. itself can be prepared, as well as various monoalkyl, dialkyl, trialkyl, and tetraalkyl ammonium salts. Also, one or more of the alkyl groups of such ammonium salts can be further substituted with groups such as hydroxy groups, to provide an ammonium salt of an alkanol amine. Ammonium salts derived from diamines such as 1,2-diaminoethane are contemplated herein. The hexa-sodium salt of suramin is useful herein.

The pharmaceutically acceptable salts, esters, solvates, and prodrugs of suramin can be prepared from the parent compound by conventional chemical methods. Generally, the salts can be prepared by reacting the free acid form of the compound with a stoichiometric amount of the appropriate base in water or in an organic solvent, or in a mixture of the two; generally, non-aqueous media like ether, ethyl acetate, ethanol, isopropanol, or acetonitrile are preferred. The esters of suramin can be prepared by reacting the parent compound with an alcohol, and removal of water formed from the reaction. Alternatively, other methods can be used. Anywhere from one up to all six of the sulfonate groups of suramin can be esterified to form a mono-ester up to a hexa-ester sulfonate.

The solvates of suramin means that one or more solvent molecules are associated with one or more molecules of suramin, including fraction solvates such as, e.g., 0.5 and 2.5 solvates. The solvents can be selected from a wide range of solvents including water, ethanol, isopropanol, and the like. The prodrugs of suramin can be prepared using convention chemical methods, depending on the prodrug chosen. A prodrug is a medication or compound that, after administration, is metabolized (i.e., converted within the body) into a pharmacologically active drug. Prodrugs can be designed to improve bioavailability when a drug itself is poorly absorbed from the gastrointestinal tract. Prodrugs are intended to include covalently bonded carriers that release an active parent drug of the present invention in vivo when such prodrug is administered. In some classifications, esters are viewed as prodrugs, such as the esters of suramin described herein. Other types of prodrugs can include sulfonamide derivatives and anhydrides.

Furthermore, the various esters and prodrugs can include further derivatization to make polyethylene glycol (PEG) and polypropylene glycol (PPG) derivatives and mixed derivatives, an example of which would a pegylated derivative.

### Relevance of Transgenic Mouse Models

The use of transgenic mouse models for studying nervous system disorders such as Fragile X Syndrome and Autism Spectrum Disorder is well-established.

Fragile X Syndrome (FXS) is a neurodevelopmental disorder with a prevalence of 1 in 4000 males and 1 in 8000 females. FXS is caused by the expansion of the CGG triplet repeat within the Fragile X Mental Retardation 1 (Fmr1) gene on the X chromosome. This chain encodes for the Fragile X Mental Retardation Protein (FMRP). If there are >200 repeats of CGG, this results in hypermethylation of Fmr1 mRNA and reduced FMRP expression resulting in a wide variety of cognitive and behavioral problems as well as abnormal physical features. FXS is typically characterized by mild-to-moderate intellectual disability, anxiety, hyperactivity, seizures, social phobia, and features of autism. The physical features may include an elongated face, large or protruding ears, high arched palate, flexible finger joints, and enlarged testicles (in males) and premature ovarian failure (in females). FXS is one of the leading genetic causes of autism spectrum disorder.

Fragile X-associated tremor/ataxia syndrome (FXTAS) is a rare, genetic neurodegenerative disorder that is related to FXS. The prevalence of FXTAS is unknown but it usually affects males over 50 years old with females comprising only a small percentage of the FXTAS population. Individuals with FXTAS have a mutation in the Fmr1 gene CGG triplet repeat. Normally, this CGG triplet is repeated from 5 to about 40 times. In people with FXTAS, however, the CGG segment is repeated 55 to 200 times. This mutation is known as an FMR1 gene premutation. FXTAS affects the neurologic system and progression is variable. Symptoms may include memory loss, slowed speech, tremors, and a shuffling gait. Some people with FXTAS show a step-wise progression (i.e., symptoms plateau for a period of time but then suddenly get worse) with acute illnesses, major surgery, or other major life stressors causing symptoms to worsen more rapidly.

Autism Spectrum Disorder (ASD) is a group of neurodevelopmental disorders with a wide variety of symptoms. ASD is one of the most common pervasive developmental disorders with a prevalence of approximately 1% worldwide. ASD has a strong genetic component but is a very heterogenous disorder with no single gene mutation responsible for more than 1-2% of cases. It is characterized by impairments in social interaction and communication across multiple contexts as well as restricted and repetitive patterns of behavior. It is often accompanied by sensory and motor abnormalities, sleep disturbances, anxiety, attention deficit hyperactivity disorder (ADHD), intellectual disabilities, and aggression.

In 1994 a consortium of Dutch and Belgian scientists developed a mouse model for FXS in which the Fmr1 gene was inactivated. These Fmr1 knockout mice lacked normal Fmr1 RNA and normal levels of FMRP which are crucial for normal CNS development. The mice exhibit impaired cognitive function including learning problems (particularly in spatial learning and associative learning), abnormal social behavior, increased locomotor activity, and male mice have enlarged testes. Fmr1 knockout mice exhibit many phenotypic and anatomic similarities to people with diagnoses of FXS and ASD. People with FXS and ASD and Fmr1 knockout mice all show high levels of anxiety-like behavior, cognitive and learning impairments, deficits in sensory gating and increase susceptibility to seizures, and sleep problems. FMRP has been suggested to regulate the length of the circadian period and abnormal sleep patterns are observed in Fmr1 knockout mice as well as people with FXS and ASD. In addition, male Fmr1 knockout mice exhibit enlarged testicles which are often observed in males with FXS. Finally, there are also abnormalities in the mouse neuronal dendritic spine morphology, like that observed in humans with FXS.

The Fmr1 knockout mouse model demonstrates many of the same cognitive and behavioral phenotypes and some anatomical features commonly observed in FXS and ASD. The development of this mouse model has furthered our understanding of several molecular and synaptic deficits underlying FXS, including abnormal dendritic spine morphology, protein dysregulation and neurotransmission. It is an excellent model for better understanding the etiology and underlying mechanisms of FXS and ASD and are a valuable tool for testing new pharmacological treatments. While all animal models have some limitations, this one closely replicates the cognitive, behavioral, and in some cases, anatomic phenotypes for both FXS and ASD. It is a well-established and well-accepted model for investigators and scientists working in neurodevelopmental disorders.

See,
Fmr1 knockout mice: a model to study fragile X mental retardation. The Dutch-Belgian Fragile X Consortium. Cell. 1994; 78(1):23-33;
Comely T A, Harris J B, Willems P J, et al. Abnormal dendritic spines in fragile X knockout mice: maturation and pruning deficits. Proc Natl Acad Sci USA. 1997; 94(10):5401-5404. doi:10.1073/pnas.94.10.5401;
Kazdoba T M, Leach P T, Silverman J L, Crawley J N. Modeling fragile X syndrome in the Fmr1 knockout mouse. Intractable Rare Dis Res. 2014; 3(4):118-133. doi:10.5582/irdr.2014.01024;
Won H, Mah W, Kim E. Autism spectrum disorder causes, mechanisms, and treatments: focus on neuronal synapses. Front Mol Neurosci. 2013; 6:19. Published 2013 Aug. 5. doi:10.3389/fnmol.2013.00019; and
Zafarullah M, Tassone F. Fragile X-Associated Tremor/Ataxia Syndrome (FXTAS). Methods Mol Biol. 2019; 1942:173-189. doi:10.1007/978-1-4939-9080-1_15.

Dosages

For the present invention for treating nervous system disorders, dosages of suramin in the compositions administered will be in the range of about 0.01 mg to about 200 mg per dose, or about 0.01 mg to about 100 mg per dose, such as a dose of a nasal spray, based on the suramin active, where each administered spray dose would comprise about 0.1 ml of liquid.

Compositions can also be determined on a weight basis. In one embodiment the compositions useful here comprise from about 0.01% to about 60% by weight suramin or a pharmaceutically salt, ester, solvate or, prodrug thereof, based on the weight of the suramin active. In another embodiment these compositions here comprise from about to about 25% by weight suramin or a pharmaceutically salt, ester, solvate or, prodrug thereof, based on the weight of the suramin active

For these foregoing compositions comprising a designated amount or weight percentage of the suramin, the suramin is determined or calculated based on the actual amount of the suramin moiety, which has a molar mass of 1297.26 grams/mole, and not including the additional weight provided by any counter ions, or ester, solvate or prodrug moieties when a suramin salt, ester, solvate, or prodrug is used. In other words, the compositions are based on the amount or weight percentage of the suramin chemical moiety.

Furthermore, because the present invention is related to intranasal delivery compositions and because it is highly desirable to limit systemic exposure, the unit dosage could be formulated to limit the systemic plasma levels of the suramin. Generally, it would be desirable to maintain the suramin plasma levels below a concentration of about 3 micromolar. In further embodiments it would be desirable to maintain the suramin plasma levels below a concentration of about 2 micromolar. In further embodiments it would be desirable to maintain the suramin plasma levels below a concentration of about 1 micromolar. In further embodiments it would be desirable to maintain the suramin plasma levels below a concentration of about 0.1 micromolar. In further embodiments it would be desirable to maintain the suramin plasma levels below a concentration of about 0.05 micromolar. In further embodiments it would be desirable to maintain the suramin plasma levels below a concentration of about 0.01 micromolar. Although a minimum systemic suramin plasma level may not be necessary if the appropriate brain blood and tissue levels are maintained, it may generally be desirable that the suramin plasma levels be greater than about 1 nanomolar.

Furthermore, because the present invention is related to intranasal compositions and methods of treatment it is highly desirable to limit systemic exposure of the suramin to minimize the potential for drug toxicity and undesired side effects and to maintain an appropriate window of safety. This limitation of systemic levels can be achieved by controlling the PK/PD profile. In some embodiments, the unit dosage should demonstrate at least one of the following blood plasma pharmacokinetic parameters for delivery of that unit dosage: a Cₘₐₓ less than about 75 micromolar (i.e. μM), or less than about 7.5 micromolar, or less than about 0.1 micromolar, or an AUC less than about 80 μg*day/L, or less than about 75 μg*day/L, or less than about 50 μg*day/L, or less than about 25 μg*day/L, or less than about 10 μg*day/L. The Cₘₐₓ can be above at least about 0.01 micromolar. The Cₘₐₓ values can be converted from micromolar to ng/ml (based on the suramin active using a molecular weight of 1297.26 grams/mole) meaning that 1 micromolar is equivalent to 1297.26 ng/ml. Should one want to have the amount based on the hexa-sodium salt a value of 1429.15 grams/mole can be used for the conversion calculation.

Methods of Treatment and Dosing Regimens

The present invention utilizes a therapeutically effective amount of suramin or a pharmaceutically acceptable salt thereof and a pharmaceutically acceptable carrier for intranasally administering suramin for treating a nervous system disorder such as ASD, FXS, FXTAS, ME/CFS, PTSD, TS, PD, AS, or the CNS disorder manifestations associated with Lyme disease, COVID-19, other viruses (e.g. Epstein Barr Human Herpesvirus 6 or 7, Herpes Simplex Virus, Cytomegalovirus, and others), including their long term effects.

The methods comprise intranasally administering a therapeutically effective amount of suramin, or a pharmaceutically acceptable salt, ester, solvate, or prodrug thereof to a human patient, in need thereof.

Various dosing regimens can be prescribed and used based on the skill and knowledge of the physician or other practitioner. In some embodiments, a unit dosage of the composition, as described herein can be applied at least once daily. In other embodiments, a unit dosage of the composition can be applied at least twice daily, or at least once weekly, or at least twice weekly. Based on the pharmacokinetic and pharmacodynamic parameters of suramin, the dosing amount and regimen can be appropriately varied. Suramin is approximately 99-98% protein bound in the serum and has a half-life of 41-78 days with an average of 50 days.

Therapy can be continued in the judgment of the physician or practitioner until the desired therapeutic benefit is achieved. In some instances, it can be desirable to continue long term or maintenance therapy.

Evaluation of Treatments

The present invention provides a method wherein the nervous system disorder, such as ASD, FXS, FXTAS, ME/CFS, PTSD, TS, PD, AS, or the CNS disorder manifestations associated with Lyme disease, COVID-19, other viruses (e.g. Epstein Barr Human Herpesvirus 6 or 7, Herpes Simplex Virus, Cytomegalovirus, and others), including their long term effects includes one or more symptoms selected from difficulty communicating, difficulty interacting with others, disruptive and repetitive behaviors, motor tics, and phonic tics. With these disorders, the patient can often exhibit one or more symptoms or behavioral manifestations, or study endpoints selected from the group consisting of
a) anxiety or anxiety-like behavior,
b) willingness to explore the environment,
c) social interaction,
d) spatial learning and memory,
e) learning and memory,
f) irritability, agitation and or crying,
g) lethargy and/or social withdrawal,
h) stereotypic behavior,
i) hyperactivity and/or noncompliance, or
j) restrictive and/or repetitive behaviors.

Patients with ASD, FXS, FXTAS, ME/CFS, PTSD, TS, PD, AS, or the CNS disorder manifestations associated with Lyme disease, COVID-19, other viruses (e.g. Epstein Barr Human Herpesvirus 6 or 7, Herpes Simplex Virus, Cytomegalovirus, and others), including their long term effects can be evaluated using a variety of rating scales to determine the level of severity of their disorder and any improvements or changes upon administration of a treatment.

For example, the present invention provides a method wherein treating the ASD, FXS, FXTAS, ME/CFS, PTSD, TS, PD, AS, or the CNS disorder manifestations associated with Lyme disease, COVID-19, other viruses (e.g. Epstein Barr Human Herpesvirus 6 or 7, Herpes Simplex Virus, Cytomegalovirus, and others), including their long term effects comprises improving more or more symptoms of the patient relative to the symptoms prior to therapy. The improvement can be determined by comparing an assessment score of the patient's symptoms relative to a score from the patient's symptoms prior to said administration. It is desirable to provide an improvement relative to a score from the patient prior to administration of the treatment. In some embodiment, it is desirable to provide an improvement of 10% or more relative to a score from the patient prior to administration of the treatment.

Examples of assessment scales for evaluating autism spectrum disorder include those selected from ABC, ADOS, ATEC, CARS CGI, and SRS.

The term “ABC” is also known as the “Aberrant Behavior Checklist” and is a rating scale for evaluating autism. The term “ADOS” is also known as “The Autism Diagnostic Observation Schedule”. The protocol consists of a series of structured and semi-structured tasks that involve social interaction between the examiner and the person under assessment. The term “ATEC” is also known as “The Autism Treatment Evaluation Scale” and is a 77-item diagnostic assessment tool that was developed at the Autism Research Institute. The ATEC was originally designed to evaluate the effectiveness of autism treatments, but is also used as a screening tool. The term “CARS” is also known as “The Childhood Autism Rating Scale” and is a behavior rating scale intended to help diagnose and evaluate autism. The term “CGI” is also known as “The Clinical Global Impression” rating scale and is a measure of symptom severity, treatment response and the efficacy of treatments in treatment studies of patients with psychological disorders. The term “SRS” is also known as the “Social Responsiveness Scale” which is used herein and is a measure of autism spectrum disorder.

For example, the present invention provides a method wherein an ADOS score of the patient is improved by 1.6 or more relative to a score prior to administration of treatment, or a corresponding performance improvement on a similar test. Furthermore, the present invention provides a method wherein the p-value of improvement of ADOS score or similar test is 0.05 or less. In another aspect, the present invention provides a method wherein the size effect of improvement of the ADOS score or similar test is about 1 or more or is up to about 2.9 or more.

Formulations for Intranasal Administration and Penetration Enhancers

The target indication of the invention compositions are nervous system disorders such as ASD, FXS, FXTAS, ME/CFS, PTSD, TS, PD, AS, or the CNS disorder manifestations associated with Lyme disease, COVID-19, other viruses (e.g. Epstein Barr Human Herpesvirus 6 or 7, Herpes Simplex Virus, Cytomegalovirus, and others), including their long term effects. As such, efforts are made to provide formulations that can readily reach the brain areas by crossing the blood-brain barrier. A feasible route of administration is delivery via the nasal cavity by a nasal drug delivery system, i.e. an intranasal (IN) formulation.

Useful compositions for intranasal delivery can be in the form of nasal sprays, liquids, powders, gels, ointments, creams, foams, aerosols, and nebulizers, among other possibilities. These compositions can have the active in the form of aqueous compositions. In other embodiments, the active agent can be a fine powder, and further in combination with particulate dispersants and diluents, or alternatively combined to form or coat the particulate dispersants. These compositions would generally be on the order of about 0.01 ml to about 0.5 ml, with a target volume of about 0.1 ml per spray, when the composition is in the form a liquid nasal spray. One to two sprays could be applied to provide a unit dosage.

The pharmaceutical compositions herein can comprise a penetration enhancer. Surprisingly, the following penetration enhancers have been found to increase the transmucosal tissue penetration of suramin: methyl Beta-cyclodextrin, caprylocaproyl macrogol-8 glycerides, and 2-(2-ethoxyethoxy)ethanol. The material methyl Beta-cyclodextrin (methyl-beta-cyclodextrin) is also known by the CAS Registry Number 128446-36-6 and the trade name methyl betadex. The material caprylocaproyl macrogol-8 glycerides is also known as caprylocaproyl polyoxyl-8 glycerides and PEG-8 caprylic/capric glycerides, by the CAS Registry Number 85536-07-8, and the trade name Labrasol®. The material 2-(2-ethoxyethoxy)ethanol is also known as diethylene glycol ethyl ether, by the CAS Registry Number 111-90-0, and by the trade names Carbitol™ and Transcutol® P.

The penetration enhance is generally used at about 40% by weight of the composition. Other useful ranges are from about 0.1% to about 90% by weight of the composition, or from about 1% to about 80% by weight of the composition, or from about 10% to about 75% by weight of the composition, or from about 25% to about 50% by weight of the composition.

The water in the composition is usually Q.S. The abbreviation QS stands for Quantum Satis and means to add as much of the ingredient, in this case water, to achieve the desired result, but not more.

Other ingredients can include various salts for osmolality control and thickening agents.

In some embodiment compositions can comprise the following functional ingredients:
1. Active ingredient: suramin, in concentration of 10 to 200 mg/mL
2. A solvent/carrier, e.g. water
3. A tissue permeation enhancer
4. A preservative(s)
5. A thickener to modify the spray solution viscosity, and
6. A buffering (pH adjusting) or osmolarity agent.

These formulations can be made using standard formulation and mixing techniques familiar to one of ordinary skill in the art of pharmaceuticals and formulations.

In one embodiment, the compositions or formulations of the present invention comprise suramin or a pharmaceutically acceptable salt, ester, solvate, or prodrug thereof and a pharmaceutically acceptable carrier. These formulations can be made using standard formulation and mixing techniques familiar to one of ordinary skill in the art of pharmaceuticals and formulations.

In one aspect, the pharmaceutical composition is selected from a solution, suspension, or dispersion for administration as a spray or aerosol. In other aspects the formulation can be delivered as drops by a nose dropper or applied directly to the nasal cavity. Other pharmaceutical compositions are selected from the group consisting of a gel, ointment, lotion, emulsion, cream, foam, mousse, liquid, paste, jelly, or tape, that is applied to the nasal cavity.

Useful herein are compositions wherein the pharmaceutically acceptable carrier is selected from water or mixtures of water with other water-miscible components. In the case of emulsions, the components do not have to be miscible with water.

In other embodiments the compositions can comprise a buffer to maintain the pH of the drug formulation, a pharmaceutically acceptable thickening agent, humectant and surfactant. Buffers that are suitable for use in the present invention include, for example, hydrochloride, acetate, citrate, carbonate and phosphate buffers.

The viscosity of the compositions of the present invention can be maintained at a desired level using a pharmaceutically acceptable thickening agent. Thickening agents that can be used in accordance with the present invention include for example, xanthan gum, carbomer, polyvinyl alcohol, alginates, acacia, chitosans, sodium carboxyl methylcellulose (Na CMC) and mixtures thereof. The concentration of the thickening agent will depend upon the agent selected and the viscosity desired.

In other embodiments, the compositions can be in the form of mucoadhesive sprayable gels. Although the nasal mucosa represents an excellent route for administration of the suramin, the protective features of the mucous secretions can make delivery challenging. It is therefore found that a mucoadhesive gel, that can be applied as a sprayed formulation provides a means of delivery. However, the gels must have the appropriate fluid characteristics to be packaged into and delivered from a spray device, such as to demonstrate shear thinning. The resultant gels must also possess the appropriate viscosity and gelling capacity. Particularly useful for delivering the appropriate spray characteristics are high acyl gellan gums. Gellan gums are water-soluble anionic polysaccharides produced by the bacterium Sphingomonas elodea and identified by the CAS Registry number 71010-52-1. Other gellant materials can also be employed provided they provide the desired rheological properties. High acyl gellan gums are commercially available as Gellan Gum LT100 from Modernist Pantry, Gellan Gum E418 high acyl (HA) from Cinogel Biotech, and Kelcogel™ from CP Kelco (USA).

The compositions of the present invention also include a tolerance enhancer to reduce or prevent drying of the mucus membrane (humectants) and to prevent irritation thereof. Suitable tolerance enhancers that can be used in the present invention include, for example, humectants, sorbitol, propylene glycol, mineral oil, vegetable oil and glycerol; soothing agents, membrane conditioners, sweeteners and mixtures thereof. The concentration of the tolerance enhancer(s) in the present compositions will also vary with the agent selected.

To enhance absorption of the drug through the nasal mucosa, a therapeutically acceptable surfactant may be added to the intranasal formulation. Suitable surfactants that can be used in accordance with the present invention include, for example, polyoxyethylene derivatives of fatty acid partial esters of sorbitol anhydrides, such as for example, Tween 80, Polyoxyl 40 Stearate, Polyoxy ethylene 50 Stearate, fusidates, bile salts and Octoxynol. Suitable surfactants include non-ionic, anionic and cationic surfactants. These surfactants can be present in the intranasal formulation in a concentration ranging from about 0.001% to about 20% by weight.

In the present invention other optional ingredients may also be incorporated into the nasal delivery system provided they do not interfere with the action of the drug or significantly decrease the absorption of the drug across the nasal mucosa. Such ingredients can include, for example, pharmaceutically acceptable excipients and preservatives. The excipients that can be used in accordance with the present invention include, for example, bio-adhesives and/or swelling/thickening agents.

In the present invention, any other suitable absorption enhancers as known in the art may also be used.

Preservatives can also be added to the present compositions. Suitable preservatives that can be used with the present compositions include, for example, benzyl alcohol, parabens, thimerosal, chlorobutanol and benzalkonium, with benzalkonium chloride being preferred. Typically, the preservative will be present in the present compositions in a concentration of up to about 2% by weight. The exact concentration of the preservative, however, will vary depending upon the intended use and can be easily ascertained by one skilled in the art.

The absorption enhancing agent includes (i) a surfactant; (ii) a bile salt (including sodium taurocholate); (iii) a phospholipid additive, mixed micelle, or liposome; (iv) an alcohol (including a polyol as discussed above, for example, propylene glycol or polyethylene glycol such as PEG 3000, etc.); (v) an enamine; (vi) a nitric oxide donor compound; (vii) a long-chain amphipathic molecule; (viii) a small hydrophobic uptake enhancer; (ix) sodium or a salicylic acid derivative; (x) a glycerol ester of acetoacetic acid; (xi) a cyclodextrin or cyclodextrin derivative; (xii) a medium-chain or short-chain (e.g. CI to C 12) fatty acid; and (xiii) a chelating agent; (xiv) an amino acid or salt thereof; and (xv) an N-acetylamino acid or salt thereof.

Solubility enhancers may increase the concentration of the drug or pharmaceutically acceptable salt thereof in the formulation. Useful solubility enhancers include, e.g., alcohols and polyalcohols.

An isotonizing agent may improve the tolerance of the formulation in a nasal cavity. A common isotonizing agent is NaCl. Preferably, when the formulation is an isotonic intranasal dosage formulation, it includes about 0.9% NaCl (v/v) in the aqueous portion of the liquid carrier.

The thickeners may improve the overall viscosity of the composition, preferably to values close to those of the nasal mucosa. Suitable thickeners include methylcellulose, carboxymethylcellulose, polyvinylpyrrolidone, sodium alginate, hydroxypropylmethylcellulose, and chitosan.

A humectant or anti-irritant improves the tolerability of the composition in repeated applications. Suitable compounds include, e.g. glycerol, tocopherol, mineral oils, and chitosan.

Various additional ingredients can be used in the compositions of the present invention. The compositions can comprise one or more further ingredients selected from a preservative, an antioxidant, an emulsifier, a surfactant or wetting agent, an emollient, a film-forming agent, or a viscosity modifying agent. These components can be employed and used at levels appropriate for the formulation based on the knowledge of one with ordinary skill in the pharmaceutical and formulation arts. The amounts could range from under 1 percent by weight to up to 90 percent or even over 99 percent by weight.

In one aspect, a preservative can be included. In another aspect, an antioxidant can be included. In another aspect, an emulsifier can be included. In another aspect, an emollient can be included. In another aspect, a viscosity modifying agent can be included. In another aspect, a surfactant or wetting agent can be included. In another aspect, a film forming agent can be included. In another aspect, the pharmaceutical composition is in the form selected from the group consisting of a gel, ointment, lotion, emulsion, cream, liquid, spray, suspension, jelly, foam, mousse, paste, tape, dispersion, aerosol. These components can be employed and used at levels appropriate for the formulation based on the knowledge of one with ordinary skill in the pharmaceutical and formulation arts.

It has surprisingly been found that penetration enhancers such as methyl Beta-cyclodextrin, caprylocaproyl macrogol-8 glycerides, and 2-(2-ethoxyethoxy)ethanol are particularly useful for preparing an intranasal suramin formulation having improved penetration of mucosal tissue.

In another aspect, the at least one preservative can be selected from the group consisting of parabens (including butylparabens, ethylparabens, methylparabens, and propylparabens), acetone sodium bisulfite, alcohol, benzalkonium chloride, benzethonium chloride, benzoic acid, benzyl alcohol, boric acid, bronopol, butylated hydroxyanisole, butylene glycol, calcium acetate, calcium chloride, calcium lactate, cetrimide, cetylpyridinium chloride, chlorhexidine, chlorobutanol, chlorocresol, chloroxylenol, cresol, edetic acid, glycerin, hexetidine, imidurea, isopropyl alcohol, monothioglycerol, pentetic acid, phenol, phenoxyethanol, phenylethyl alcohol, phenylmercuric acetate, phenylmercuric borate, phenylmercuric nitrate, potassium benzoate, potassium metabisulfite, potassium nitrate, potassium sorbate, propionic acid, propyl gallate, propylene glycol, propylparaben sodium, sodium acetate, sodium benzoate, sodium borate, sodium lactate, sodium metabisulfite, sodium propionate, sodium sulfite, sorbic acid, sulfur dioxide, thimerosal, zinc oxide, and N-acetylcysteine, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one antioxidant can be selected from the group consisting of acetone sodium bisulfite, alpha tocopherol, ascorbic acid, ascorbyl palmitate, butylated hydroxyanisole, butylated hydroxytoluene, citric acid monohydrate, dodecyl gallate, erythorbic acid, fumaric acid, malic acid, mannitol, sorbitol, monothioglycerol, octyl gallate, potassium metabisulfite, propionic acid, propyl gallate, sodium ascorbate, sodium formaldehyde sulfoxylate, sodium metabisulfite, sodium sulfite, sodium thiosulfate, sulfur dioxide, thymol, vitamin E polyethylene glycol succinate, and N-acetylcysteine, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one emulsifier can be selected from the group consisting of acacia, agar, ammonium alginate, calcium alginate, carbomer, carboxymethylcellulose sodium, cetostearyl alcohol, cetyl alcohol, cholesterol, diethanolamine, glyceryl monooleate, glyceryl monostearate, hectorite, hydroxypropyl cellulose, hydroxypropyl starch, hypromellose, lanolin, lanolin alcohols, lauric acid, lecithin, linoleic acid, magnesium oxide, medium-chain triglycerides, methylcellulose, mineral oil, monoethanolamine, myristic acid, octyldodecanol, oleic acid, oleyl alcohol, palm oil, palmitic acid, pectin, phospholipids, poloxamer, polycarbophil, polyoxyethylene alkyl ethers, polyoxyethylene castor oil derivatives, polyoxyethylene sorbitan fatty acid esters, polyoxyethylene stearates, polyoxyl 15 hydroxystearate, polyoxylglycerides, potassium alginate, propylene glycol alginate, propylene glycol dilaurate, propylene glycol monolaurate, saponite, sodium borate, sodium citrate dehydrate, sodium lactate, sodium lauryl sulfate, sodium stearate, sorbitan esters, starch, stearic acid, sucrose stearate, tragacanth, triethanolamine, tromethamine, vitamin E polyethylene glycol succinate, wax, and xanthan gum, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one emollient can be selected from the group consisting of almond oil, aluminum monostearate, butyl stearate, canola oil, castor oil, cetostearyl alcohol, cetyl alcohol, cetyl palmitate, cholesterol, coconut oil, cyclomethicone, decyl oleate, diethyl sebacate, dimethicone, ethylene glycol stearates, glycerin, glyceryl monooleate, glyceryl monostearate, isopropyl isostearate, isopropyl myristate, isopropyl palmitate, lanolin, lanolin alcohols, lecithin, mineral oil, myristyl alcohol, octyldodecanol, oleyl alcohol, palm kernel oil, palm oil, petrolatum, polyoxyethylene sorbitan fatty acid esters, propylene glycol dilaurate, propylene glycol monolaurate, safflower oil, squalene, sunflower oil, tricaprylin, triolein, wax, xylitol, zinc acetate, or a combination thereof. These components can be employed and used at levels appropriate for the formulation based on the knowledge of one with ordinary skill in the pharmaceutical and formulation arts. The amounts could range from under 1 percent by weight to up to 60 percent by weight.

In another aspect, the at least one viscosity modifying agent can be selected from the group consisting of acacia, agar, alginic acid, aluminum monostearate, ammonium alginate, attapulgite, bentonite, calcium alginate, calcium lactate, carbomer, carboxymethylcellulose calcium, carboxymethylcellulose sodium, carrageenan, cellulose, ceratonia, ceresin, cetostearyl alcohol, cetyl palmitate, chitosan, colloidal silicon dioxide, corn syrup solids, cyclomethicone, ethylcellulose, gelatin, glyceryl behenate, guar gum, hectorite, hydrophobic colloidal silica, hydroxyethyl cellulose, hydroxyethylmethyl cellulose, hydroxypropyl cellulose, hydroxypropyl starch, hypromellose, magnesium aluminum silicate, maltodextrin, methylcellulose, myristyl alcohol, octyldodecanol, palm oil, pectin, polycarbophil, polydextrose, polyethylene oxide, polyoxyethylene alkyl ethers, polyvinyl alcohol, potassium alginate, propylene glycol alginate, pullulan, saponite, sodium alginate, starch, sucrose, sugar, sulfoburylether β-cyclodextrin, tragacanth, trehalose, and xanthan gum, or a combination thereof. These components can be employed and used at levels appropriate for the formulation based on the knowledge of one with ordinary skill in the pharmaceutical and formulation arts. The amounts could range from under 1 percent by weight to up to 60 percent.

In another aspect, the at least one film forming agent can be selected from the group consisting of ammonium alginate, chitosan, colophony, copovidone, ethylene glycol and vinyl alcohol grafted copolymer, gelatin, hydroxypropyl cellulose, hypromellose, hypromellose acetate succinate, polymethacrylates, poly(methyl vinyl ether/maleic anhydride), polyvinyl acetate dispersion, polyvinyl acetate phthalate, polyvinyl alcohol, povidone, pullulan, pyroxylin, and shellac, or a combination thereof. These components can be employed and used at levels appropriate for the formulation based on the knowledge of one with ordinary skill in the pharmaceutical and formulation arts. The amounts could range from under 1 percent by weight to up to about 90 percent or even over 99 percent by weight.

In another aspect, the at least one surfactant or wetting agent can be selected from the group consisting of docusate sodium, phospholipids, sodium lauryl sulfate, benzalkonium chloride, cetrimide, cetylpyridinium chloride, alpha tocopherol, glyceryl monooleate, myristyl alcohol, poloxamer, polyoxyethylene alkyl ethers, polyoxyethylene castor oil derivatives, polyoxyethylene sorbitan fatty acid esters, polyoxyethylene stearates, polyoxyl 15 hydroxystearate, polyoxylglycerides, propylene glycol dilaurate, propylene glycol monolaurate, sorbitan esters, sucrose stearate, tricaprylin, and vitamin E polyethylene glycol succinate, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, a buffering agent can be included. In another aspect, an emollient can be included. In another aspect, an emulsifying agent can be included. In another aspect, an emulsion stabilizing agent can be included. In another aspect, a gelling agent can be included. In another aspect, a humectant can be included. In another aspect, an ointment base or oleaginous vehicle can be included. In another aspect, a suspending agent can be included. In another aspect an acidulant can be included. In another aspect, an alkalizing agent can be included. In another aspect, a bioadhesive material can be included. In another aspect, a colorant can be included. In another aspect, a microencapsulating agent can be included. In another aspect, a stiffening agent can be included. These components can be employed and used at levels appropriate for the formulation based on the knowledge of one with ordinary skill in the pharmaceutical and formulation arts. The amounts could range from under 1 percent by weight to up to 90 percent or even over 99 by weight.

When the active ingredient is delivered as a powder, the powdered material is often combined with a powdered dispersant. In other embodiments the active can be combined with the dispersant to form particles containing both the active and the dispersant. In yet other embodiments, the active can be coated onto the surface of the dispersant. Examples of dispersants include a wide array of ingredients including sugars, such as lactose, glucose, and sucrose.

One of ordinary skill in the pharmaceutical and formulation arts can determine the appropriate levels of the essential and optional components of the compositions of the present invention.

Methods of preparing the suramin compositions are also intended as part of the present invention and would be apparent to one of ordinary skill in the pharmaceutical and formulation arts using standard formulation and mixing techniques.

Also provided in the present invention is a device for patient administration or self-administration of the antipurinergic agent comprising a nasal spray inhaler containing an aerosol spray formulation of the antipurinergic agent and a pharmaceutically acceptable dispersant or solvent system, wherein the device is designed (or alternatively metered) to disperse an amount of the aerosol formulation by forming a spray that contains the dose of the antipurinergic agent. In other embodiments, the inhaler can comprise the antipurinergic agent as a fine powder, and further in combination with particulate dispersants and diluents, or alternatively combined to form or coat the particulate dispersants.

### EXAMPLES

The following examples further describe and demonstrate embodiments within the scope of the present invention. The Examples are given solely for purpose of illustration and are not to be construed as limitations of the present invention, as many variations thereof are possible without departing from the spirit and scope of the invention.

### Example 1: Composition for Intranasal Delivery

The following composition is prepared using standard mixing equipment and procedures.

| Ingredient | Amount |
|---|---|
| Suramin hexa-sodium salt | 10-200 mg/ml* |
| Methyl beta-cyclodextrin | 40% weight |
| (methyl betadex) | |
| Water | QS to achieve the indicated |
| | levels of ingredients |

*Based on the suramin hexa-sodium salt having a molecular weight of 1429.15 grams/mole

The suramin sodium salt is dissolved in water with gentle mixing. The cyclodextrin is added with mixing until dissolved. The resultant solution is allowed to sit for 2 hours before using.

The composition can be packaged in a spray bottle for intranasal administration.

Alternatively, the compositions are prepared replacing the methyl βeta-cyclodextrin with an equal weight of caprylocaproyl macrogol-8 glycerides or and 2-(2-ethoxyethoxy)ethanol.

The compositions are useful for treating a nervous system disorder.

### Example 2: Composition for Intranasal Delivery

The following composition is prepared using standard mixing equipment and procedures.

| Ingredient | Amount |
|---|---|
| Suramin hexa-sodium salt | 10-200 mg/ml* |
| Methyl beta-cyclodextrin | 40% weight |
| (methyl betadex) | |
| Sodium chloride | 0.75% weight |
| Hydroxypropyl methyl cellulose | 0.1% weight |
| Water | QS to achieve the indicated |
| | levels of ingredients |

*Based on the suramin hexa-sodium salt having a molecular weight of 1429.15 grams/mole

The suramin sodium salt is dissolved in water with gentle mixing. The sodium chloride and the hydroxypropyl methyl cellulose are added with mixing. The cyclodextrin is added with mixing until dissolved. The resultant solution is allowed to sit for 2 hours before using.

The composition can be packaged in a spray bottle for intranasal administration.

Alternatively, compositions are prepared replacing the methyl βeta-cyclodextrin with an equal weight of caprylocaproyl macrogol-8 glycerides or and 2-(2-ethoxyethoxy)ethanol.

The compositions are useful for treating a nervous system disorder.

### Example 3: Mucoadhesive Sprayable Gel Formulation

The following composition is prepared using standard mixing equipment and procedures.

| Ingredient | Amount |
|---|---|
| Suramin hexa-sodium salt | 10-200 mg/ml* |
| High Acyl Gellan Gum | 0.1-1% weight |
| Water | QS to achieve the indicated |
| | levels of ingredients |

*Based on the suramin hexa-sodium salt having a molecular weight of 1429.15 grams/mole

The suramin sodium salt is dissolved in water with gentle mixing. The mixture us heated to about 40 to 90° C. and with gentle mixing the high acyl high acyl gellan gum is added. The mixture is then allowed to cool to room temperature and can be packaged in a spray bottle for intranasal administration.

The compositions are useful for treating a nervous system disorder.

### Example 4: Tissue Permeation of Suramin

Four formulations, A-D, were prepared using the methods of Examples 1 and 2 and found to be stable for at least 4 weeks at 25° C. and 60% relative humidity for three months.

Formulation A—suramin hexa-sodium salt at 100 mg/mL in water (no excipients)
Formulation B—suramin hexa-sodium salt at 100 mg/mL in water, with 40% methyl β-cyclodextrin (methyl betadex)
Formulation C—suramin hexa-sodium salt at 100 mg/mL in water, with 40% HP (hydroxyl propyl)-cyclodextrin
Formulation D—suramin hexa-sodium salt at 160 mg/mL in water (no excipients)
   The formulations also contained 0.1% of hydroxypropyl methyl cellulose (i.e. HPMC E5, from Dow Chemicals) as a solution thickening agent; and 0.75% sodium chloride as osmolarity agent.

These four formulations were evaluated in an in vitro permeation study using cultured human airway tissues (EpiAirway AIR-100, purchased from MatTek Corporation), following an established drug permeability protocol (EpiAirway™ Drug Permeation Protocol, MatTek Corporation, 2014). EpiAirway is representative of the upper airways extending from the trachea to the primary bronchi, therefore it is used to measure drug delivery from nasal formulations.

For receiver fluid preparation, one pre-warms the EpiAirway assay medium to 37° C. Using a sterile technique, one pipets 0.3 mL medium into each well of a sterile 24-well plate. Label the wells. Use 0.2 mL of donor solution on the tissues.

Permeability experiment: Following the overnight equilibration, move the cell culture inserts to the 1-hour wells and pipet the donor solution onto the tissue. Return the plates to the incubator. After 30 minutes of elapsed permeation time, move the tissues to 2-hour wells. Similarly move the tissues after 2.0, 3.0, 4.0 and 6.0 hours of total elapsed time. It will not be necessary to replenish the donor solution. Alternatively, one can completely remove the receiver solution at the appropriate time and replace with fresh, pre-warmed receiver fluid. The solutions were analyzed using HPLC and detection at 238 nm.

The following Table 1 provides the averaged accumulated amount, in mg, of suramin that has penetrated as a function of time.

TABLE 1

| | Total Accumulated Suramin (mg) | | | |
|---|---|---|---|---|
| Time | Formulation | | | |
| (hours) | A | B | C | D |
| 1 | 0.047 | 2.629 | 0.000 | 0.082 |
| 2 | 0.145 | 6.011 | 0.055 | 0.249 |
| 3 | 0.258 | 7.276 | 0.171 | 0.436 |
| 4 | 0.391 | 7.969 | 0.386 | 0.692 |
| 5 | 0.773 | 8.863 | 1.443 | 1.278 |
| 6 | 0.047 | 2.629 | 0.000 | 0.082 |

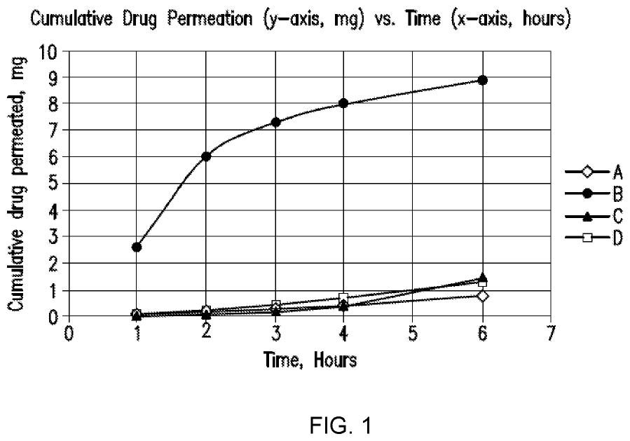

The results of the study are also shown graphically in FIG. 1 where the cumulative amount (mg) of drug permeated was plotted versus time in hours.

These data demonstrate that Formulation B containing methyl β-cyclodextrin (methyl betadex) provides significantly better penetration, versus Formulations, A, C, and D in the tissue permeation assay. Also, as is seen from a comparison of Formulations A and D, having a higher drug concentration can be advantageous to increasing permeation.

### Example 5: Tissue Permeation of Suramin

Six formulations, A-F, were prepared using the methods of Examples 1 and 2 and found to be stable for at least 4 weeks at ambient conditions.
Formulation A—suramin at 200 mg/mL in water (no excipients)
Formulation B—suramin at 140 mg/mL in water, with 40% polysorbate 80 (Tween 80)
Formulation C—suramin at 140 mg/mL in water, with 40% methyl Beta-cyclodextrin (methyl betadex)
Formulation D—suramin at 140 mg/mL in water, with 40% sulfobutylether beta-cyclodextrin (Captisol)
Formulation E—suramin at 140 mg/mL in water, with 40% 2-(2-ethoxyethoxy)ethanol (Transcutol P)
Formulation F—suramin at 140 mg/mL in water (Labrasol)

Tissue permeability studies were conducted using the methods of Example 3.

The following Table 2 provides the averaged accumulated amount, in mg, of suramin that has penetrated as a function of time.

TABLE 2
Total Accumulated Suramin (mg)

| | |
|---|---|
| Time | Formulation |

| (hours) | A | B | C | D | E | F |
|---|---|---|---|---|---|---|
| 1 | 0.09 | 0.05 | 3.69 | 0.05 | 1.47 | 3.20 |
| 2 | 0.40 | 0.39 | 12.22 | 0.45 | 5.03 | 6.77 |
| 3 | 1.01 | 0.65 | 15.57 | 1.12 | 8.67 | 8.23 |
| 4 | 2.16 | 1.08 | 19.11 | 2.41 | 13.32 | 9.74 |
| 6 | 5.93 | 1.88 | 22.24 | 5.63 | 17.90 | 13.17 |

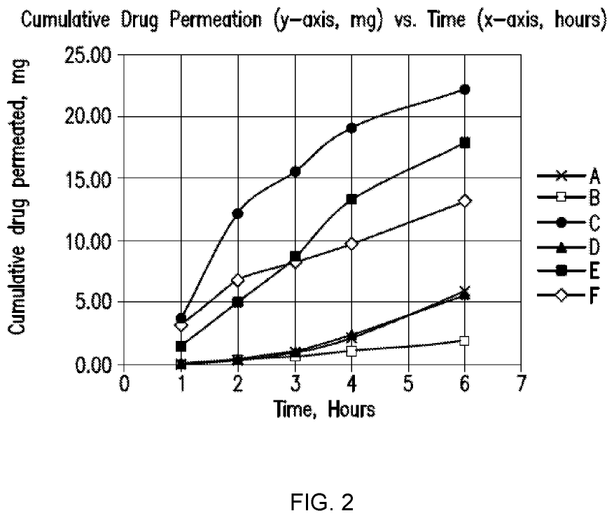

The results of the study are also showing graphically in FIG. 2 where the cumulative amount (mg) of drug permeated was plotted versus time in hours. These data demonstrate that Formulation C containing methyl Beta-cyclodextrin (methyl betadex), E containing 2-(2-ethoxyethoxy)ethanol (Transcutol P), and F containing caprylocaproyl macrogol-8 glycerides (Labrasol) provide significantly better penetration, versus Formulations, A, B, and D in the tissue permeation assay.

Furthermore, the results from Examples 3 and 4 are surprising.

Cyclodextrins are sugar molecules bound together in rings of various sizes. Specifically, the sugar units are called glucopyranosides—glucose molecules that exist in the pyranose (six-membered) ring configuration. Six, 8, or 10 glucopyranosides bind with each other to form α-, β-, and γ-cyclodextrin, respectively. Cyclodextrins form a toroid (truncated cone) configuration with multiple hydroxyl groups at each end. This allows them to encapsulate hydrophobic compounds without losing their solubility in water. Among other applications, cyclodextrins can be used to carry hydrophobic drug molecules into biological systems, as tissue permeation enhancers. It has been reported that the cyclodextrins form inclusion complexes with a variety of hydrophobic drugs thereby increasing their partitioning and solubility in the tissue membrane. Methyl Beta-cyclodextrin (betadex) is a type of cyclodextrin. Methyl betadex is used in at least one marketed intranasal product Estradiol (Aerodiol) to enhance trans-tissue permeation of the drug molecule, estradiol (MW=272.4). Because of its small size (MW=272.4), estradiol molecule can be easily encapsulated into the cyclodextrin ring, and thus enhancement of delivery into biological tissues is achieved.

However, we discovered a way in which methyl Beta-cyclodextrin could also be capable of encapsulating suramin, which is a much larger molecule than generally considered compatible. It is surprising to find the methyl betadex works for suramin. A person having ordinary skill in the art would not have been expected that such a large molecule could be encapsulated into cyclodextrin ring.

Another useful penetration enhancer is Transcutol P (Diethylene glycol mono-ethyl ether). This is an excipient which has been reported to enhancer skin permeability for some small molecule drug compounds in various topical/transdermal formulations. Nevertheless, it has not been used as an excipient for intranasal products. Also, it is not commonly used to enhance large molecule such as suramin.

Another useful penetration enhancer is Labrasol (Caprylocaproyl macrogol-8 glycerides). This is an excipient that have been reported to enhancer skin permeability of some drug compounds in some topical/transdermal formulations. It has not been used as an excipient for intranasal products.

### Example 6: Determination of Suramin in Plasma and Brain Tissue

The following example describes a mouse study conducted to determine the delivery of suramin to plasma and brain tissue when administered intraperitoneally (IP) or intranasally (IN) according to different treatment regimens. For the study, male Fmr1-knockout B6.129P2-Fmr1tm1Cgr/J TG mice were purchased from Jackson Laboratories, Bar Harbor, Maine. These mice were of approximately 8 weeks of age. These mice exhibit abnormalities of dendritic spines in multiple regions of the brain. The absence of FMRP in these mice induces an over-activation of RAC1, a protein of the Rho GTPase subfamily that plays a critical role in dendritic morphology and synaptic function. These B6.129P2-Fmr1tm1Cgr/J TG mice, provide an animal model for cognitive disabilities and neurodevelopmental disorders.

The mice were maintained in group cages (6 mice per cage based on treatment group) in a controlled environment (temperature: 21.5±4.5° C. and relative humidity: 35-55%) under a standard 12-hour light/12-hour dark lighting cycle (lights on at 06:00). Mice were accommodated to the research facility for approximately a week. Body weights of all mice were recorded for health monitoring purposes.

The mice were divided into the following 5 test groups, with 6 mice per group.
Group 1: Intraperitoneal (IP) injection of suramin, 20 mg/kg, administered weekly to animals beginning at 9 weeks of age and continuing for four weeks (i.e. given at Age Weeks 9, 10, 11 and 12). The suramin was formulated in Normal saline solution.
Group 2: Intraperitoneal (IP) injection of saline, 5 mL/g, administered weekly to animals beginning at 9 weeks of age and continuing for four weeks (i.e. given at Age Weeks 9, 10, 11 and 12). This was a control group.
Group 3: Intranasal (IN) administration of a formulation, described below, of suramin, at a concentration of 100 mg/mL×6 mL per spray, administered as one spray per nostril, one time per day, (interval of each application is around 2 minutes to ensure absorption) for 28 days (total of 56 sprays over 28 day period) beginning at 9 weeks of age (i.e. given daily during Age Weeks 9, 10, 11 and 12).
Group 4: Intranasal (IN) administration of a formulation, described below, of suramin, at a concentration of 100 mg/mL×6 mL per spray, administered as one spray per nostril, one time every other day, for 28 days (total of 28 sprays over 28 day period) beginning at 9 weeks of age (i.e. given once every other day during Age Weeks 9, 10, 11 and 12).
Group 5: Intranasal (IN) administration of a formulation, described below, of suramin, at a concentration of 100 mg/mL×6 mL per spray, administered as one spray per nostril, one time every week, for 4 weeks (28 days) (total of 8 sprays over 28 day period) beginning at 9 weeks of age (i.e. given once weekly during Age Weeks 9, 10, 11 and 12).

The following is the suramin intranasal (IN) formulation administered to Groups 3, 4, and 5, above.

| | Weight (grams) | Percent of Composition |
|---|---|---|
| Suramin hexa-sodium salt | 16.6 | 10.3% |
| Methyl beta cyclodextrin | 50 | 30.9% |
| Benzalkonium chloride | 0.04 | 0.012% |
| (50% aqueous solution) | | |
| HPMC E5* | 5.6 | 3.46% |
| Citric acid | 0.3 | 0.19% |
| Sodium sulfite | 0.15 | 0.093% |
| Water | 89.13 | 55.1% |
| Total | 161.82 | 100% |

*HPMC E5 is a water-soluble cellulose ethers polymer [hydroxypropyl methylcellulose (HPMC)] available from DuPont.

The above formulation is made by dissolving the suramin sodium salt in water with gentle mixing. The remaining ingredients, except the cyclodextrin are added with mixing. The cyclodextrin is then added with mixing until dissolved. The resultant solution is allowed to sit for 2 hours before using.

Blood samples were collected from all mice at the end of 12 weeks of age. Brain tissue was harvested from all mice upon sacrifice 13-14 weeks of age. Standard sample preparation and analytical techniques were used to obtain the data.

The results from this study are shown in Table 3. The data is presented as the average plasma concentration (in both ng/ml and μM) for each animal group and average brain tissue concentration (in both ng/g and mmol/g). Also presented is the average brain tissue to plasma partition ratio for each group. Note that such a calculation is not applicable for the group administered a saline control (Group 2) as no suramin was detected in the brain tissue and the small plasma levels are essentially noise from the analytical method.

TABLE 3

| | | | Average Brain | | Average Brain |
|---|---|---|---|---|---|
| | Average Plasma | | Tissue | | Tissue to Plasma |
| | Concentration | | Concentration | | Partitioning |
| Group | ng/ml | μM | ng/g | mmol/g | Ratio¹ |
| 1 | 18733 | 14.440 | 550 | 0.424 | 0.030 |
| 2 | 88.3 | 0.068 | BQL² | BQL² | NA³ |
| 3 | 1637 | 1.262 | 115.2 | 0.089 | 0.069 |
| 4 | 1578 | 1.217 | 127.5 | 0.098 | 0.089 |
| 5 | 278.7 | 0.215 | 91.3 | 0.070 | 0.235 |

¹The partitioning ratio is calculated directly from the raw data rather than the averages presented in the table.
²BQL means below quantifiable limit.
³NA means not applicable.

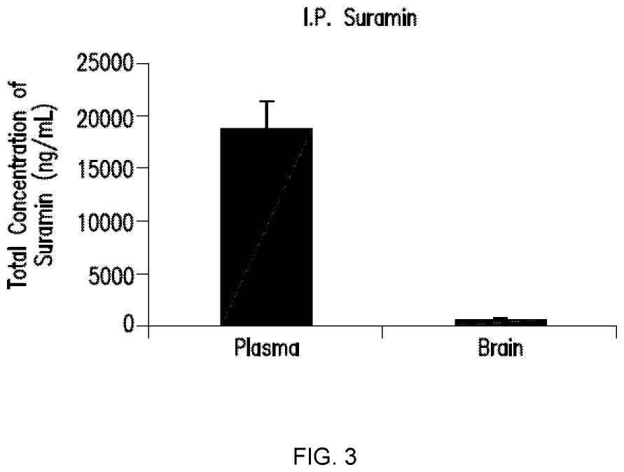

The results from the study are also shown in the plots of FIGS. 3 through 10.

FIG. 3 shows a plot of the total concentration, in ng/ml, of suramin in plasma versus brain tissue in mice when administered by intraperitoneal (IP) injection, 20 mg/kg, weekly to the mice beginning at 9 weeks of age and continuing for four weeks (i.e. given at age weeks 9, 10, 11 and 12).

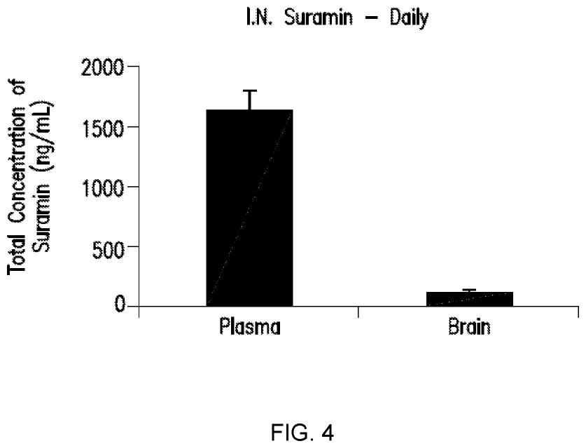

FIG. 4 shows a plot comparing the total concentration, in ng/ml, of suramin in plasma versus brain tissue in mice when administered intranasally (IN) daily for 28 days. A composition of the present invention comprising IN suramin, at a concentration of 100 mg/mL×6 mL per spray, was administered as one spray per nostril, one time per day, (interval of each application is around 2 minutes to ensure absorption) for 28 days (total of 56 sprays over 28 day period) beginning at 9 weeks of age (i.e. given daily during age weeks 9, 10, 11 and 12).

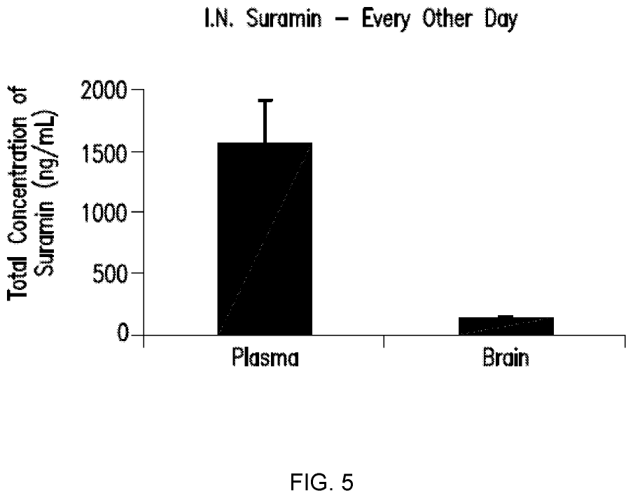

FIG. 5 shows a plot comparing the total concentration, in ng/ml, of suramin in plasma versus brain tissue in mice when administered intranasally (IN) every other day for 28 days. A composition of the present invention comprising IN suramin, at a concentration of 100 mg/mL×6 mL per spray, was administered as one spray per nostril, every other day, (interval of each application is around 2 minutes to ensure absorption) for 28 days (total of 28 sprays over 28 day period) beginning at 9 weeks of age (i.e. given daily during age weeks 9, 10, 11 and 12).

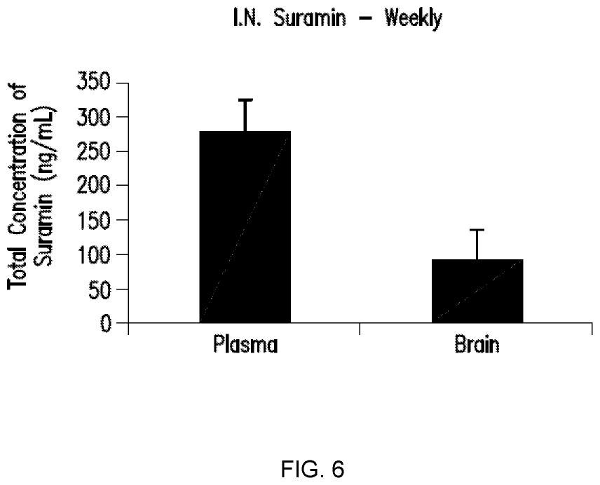

FIG. 6 shows a plot comparing the total concentration, in ng/ml, of suramin in plasma versus brain tissue in mice when administered intranasally (IN) once per week for 4 weeks. A composition of the present invention comprising IN suramin, at a concentration of 100 mg/mL×6 mL per spray, was administered as one spray per nostril, one time per week, (interval of each application is around 2 minutes to ensure absorption) for 4 weeks (28 days) (total of 8 sprays over 28 day period) beginning at 9 weeks of age (i.e. given daily during age weeks 9, 10, 11 and 12).

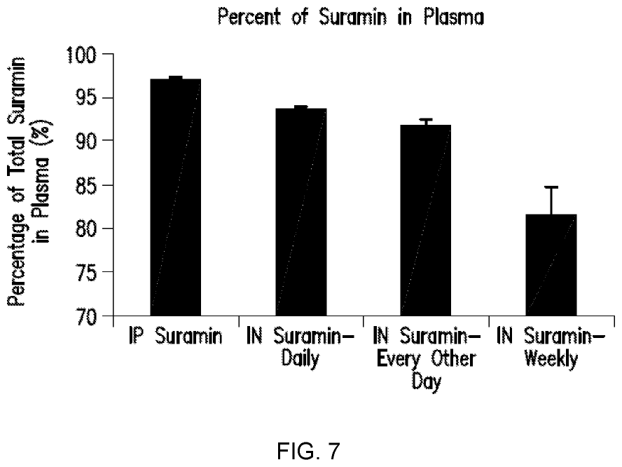

FIG. 7 shows a plot comparing the total percentage of suramin in plasma in mice when administered by intraperitoneal (IP) injection once weekly for 4 weeks (28 days), intranasally (IN) daily for 28 days, intranasally (IN) every other day for 28 days, and intranasally (IN) once per week for 4 weeks (28 days).

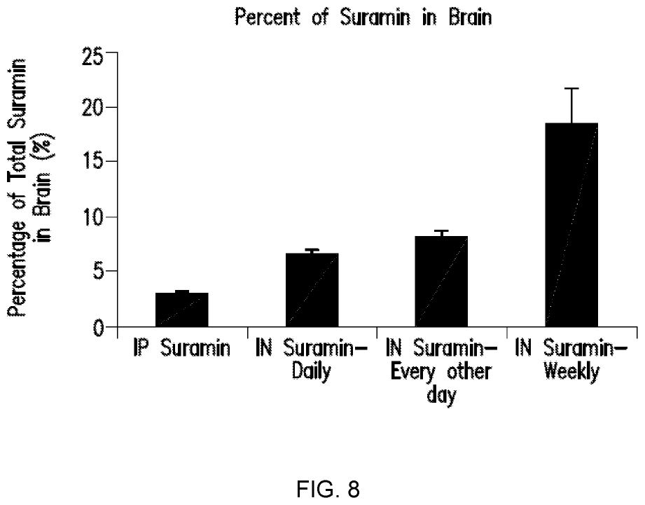

FIG. 8 shows a plot comparing the total percentage of suramin in brain tissue in mice when administered by intraperitoneal (IP) injection once weekly for 4 weeks (28 days), intranasally (IN) daily for 28 days, intranasally (IN) every other day for 28 days, and intranasally (IN) once per week for 4 weeks (28 days).

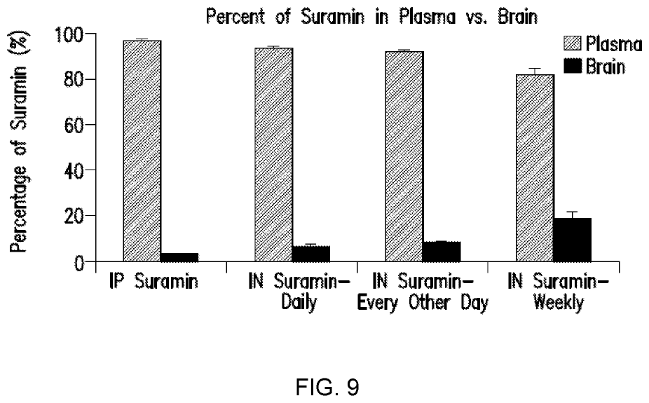

FIG. 9 shows a plot comparing the total percentage of suramin in plasma versus brain tissue in mice when administered by intraperitoneal (IP) injection once weekly for 4 weeks (28 days), intranasally (IN) daily for 28 days, intranasally (IN) every other day for 28 days, and intranasally (IN) once per week for 4 weeks (28 days).

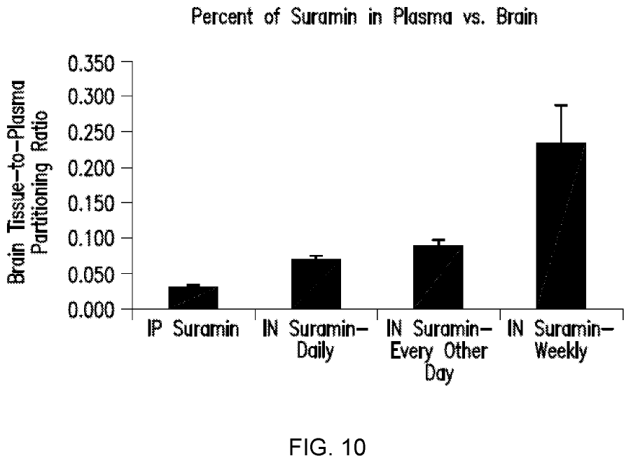

FIG. 10 shows a plot comparing the brain tissue to plasma partitioning ratio of suramin in mice when administered by intraperitoneal (IP) injection once weekly for 4 weeks (28 days), intranasally (IN) daily for 28 days, intranasally (IN) every other day for 28 days, and intranasally (IN) once per week for 4 weeks (28 days).

These results demonstrate that an antipurinergic agent such as suramin can be delivered intranasally to achieve plasma and brain tissue levels and that variations in the brain tissue to plasma partitioning ratio can be observed. These results therefore demonstrate that an antipurinergic agent such as suramin can be delivered to the brain of a mammal by intranasal (IN) administration.

### Example 7: Evaluation of Suramin in a Light/Dark Preference Test (LDT): Anxiety-Like Behavior

Objective

The purpose of this light/dark study was to test various suramin formulations and treatment routes and regimens in B6.129P2-Fmr1tm1Cgr/J transgenic (TG) mice to determine if there is an impact of these agents in ameliorating unconditioned anxiety-like behavior compared to wild type mice and TG mice treated with IP saline as controls.

### BACKGROUND

The light/dark preference test (LDT) is one of the most widely used tests in pharmacology to measure unconditioned anxiety-like behavior in mice. The test is based on the natural aversion of mice to brightly illuminated areas and on their spontaneous exploratory behavior in response to a novel environment and light. See, Takao, K., et al., Light/dark Transition Test for Mice. J. Vis. Exp. (1), e104, doi:10.3791/104 (2006).

This study used a SmartCage™ system, which is an automated non-invasive rodent behavioral monitoring system which enables biomedical researchers to conduct a variety of neurobehavioral assays through consistent and accurate monitoring of rodent home cage activity and behavior. See, Xie X. S. et al. (2012) Rodent Behavioral Assessment in the Home Cage Using the SmartCageTM™ System. In: Chen J., Xu X M., Xu Z., Zhang J. (eds) Animal Models of Acute Neurological Injuries II. Springer Protocols Handbooks. Humana Press, Totowa, NJ.

### Materials and Methods:

### Experimental Arms (Treatment Groups):

1. N=6 mice per group.
2. All behavioral testing included a group of untreated wild type controls.
3. Dosing for the various groups were as follows:
   a. Group 1: Intraperitoneal (IP) suramin, 20 mg/kg, administered weekly to animals beginning at 9 weeks of age and continuing for four weeks (i.e. given at Age Weeks 9, 10, 11 and 12).
   b. Group 2: IP saline, 5 mL/g, administered weekly to animals beginning at 9 weeks of age and continuing for four weeks (i.e. given at Age Weeks 9, 10, 11 and 12).
   c. Group 3: Formulation of Intranasal (IN) suramin, at a concentration of 100 mg/mL×6 μL per spray, administered as one spray per nostril, one time per day, (interval of each application is around 2 min to ensure absorption) for 28 days (total of 56 sprays over 28 day period) beginning at 9 weeks of age (i.e. given daily during Age Weeks 9, 10, 11 and 12).
   d. Group 4: Formulation of IN suramin, at a concentration of 100 mg/mL×6 μL per spray, administered as one spray per nostril, one time every other day, for 28 days (total of 28 sprays over 28 day period) beginning at 9 weeks of age (i.e. given once every other day during Age Weeks 9, 10, 11 and 12).
   e. Group 5: Formulation of IN suramin, at a concentration of 100 mg/mL×6 μL per spray, administered as one spray per nostril, one time every week, for 4 weeks (28 days) (total of 8 sprays over 28 day period) beginning at 9 weeks of age (i.e. given once weekly during Age Weeks 9, 10, 11 and 12).
4. Behavioral tests for all groups (Groups 1-5) began the following day after the last day of IN dosing (Weeks 13-14 of age).
5. Blood samples (PK testing) were collected from all mice at the end of 12 Weeks of age, just prior to starting the behavioral tests in Week 13.
6. Brain tissue (for biochemistry testing) was harvested from all mice upon sacrifice at the conclusion of all behavioral testing at the end of 13-14 Weeks of age.

### Animals

Male B6.129P2-Fmr1tm1Cgr/J TG mice were purchased from Jackson Laboratories, Bar Harbor, Maine. These mice were of approximately 8(+1) weeks of age. Mice were maintained in group cages (6 mice per cage based on treatment group) in a controlled environment (temperature: 21.5±4.5° C./relative humidity: 35-55%) under a standard 12-hour light/12-hour dark lighting cycle (lights on at 06:00). Mice accommodated to the research facility for the remainder of the week. Dosing began on the following Monday. Body weights of all mice were recorded for health monitoring purposes.

### Experimental Methods:

Dosing was carried out over the course of 28 days as instructed by the study sponsor.
1. Prior to the beginning of the light/dark test, a dark box (red transparent enclosure with an opening for the mouse to enter) is placed in the SmartCage™
2. An individual mouse is placed in the open/non-dark box side (“Light Zone”), with its head facing away from the dark box.
3. The mouse is then allowed to freely explore the SmartCage™ and enter the dark box “Dark Zone” at its own discretion over a 10-minute span.
4. Anxiety-like behavior is assessed based on the SmartCage™ monitoring of time spent in the Light Zone, the number of Light Zone entries, and % Time Spent in the Light Zone.
5. Data is grouped together based on treatment group (IP Suramin, IP Saline, IN Suramin-Daily, IN Suramin-Every Other Day, IN Suramin-Weekly).
6. Wildtype Control data (collected separately prior to the beginning of the dosing of the dosing of the B6.129P2-Fmr1tm1Cgr/J TG mice) was added to the final data analysis to serve as a comparison for naive, male C57BL/6 mice.

The Light-Dark Test does not require any prior training. No food or water is withheld and only natural stressors such as light are used. Four similarly calibrated SmartCages™ were used to record four mice simultaneously (example cage shown below). All Light/Dark tests were completed in one day.

Light/Dark Test Setup—Dark Box placed within the transparent home cage; home cage placed within the SmartCage™ monitoring system.

### Results:

### Dark Zone Entry Latency:

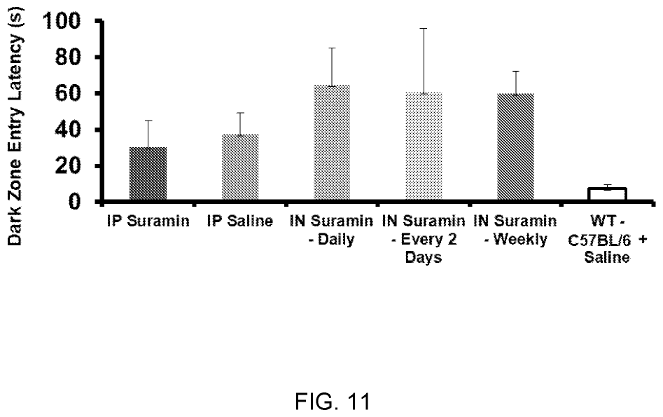

FIG. 11 shows the time to entry of the dark zone (measured in seconds). Mice could roam the SmartCage™ as well as enter and exit the dark box at their own discretion. If a mouse did not enter the dark box, that mouse was assigned an entry latency of 600 seconds (the cutoff of 10 minutes that the experiment allowed) for statistical purposes.

### Light Zone Time & Time Spent in Light Zone (%):

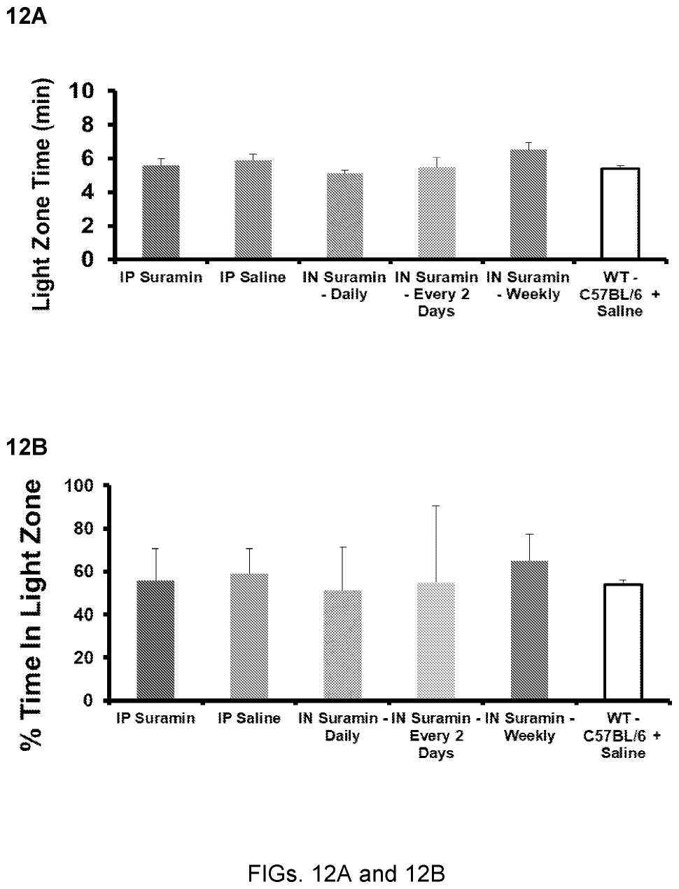

FIG. 12A shows the total time spent in the light zone (measured in minutes) and FIG. 12B shows the time spent in the light zone (expressed as a percentage). In FIG. 12A, the TG mice treated with IN Suramin-Weekly showed the longest time in the light zone (˜6.5 minutes). In assessing the total percentage of time spent in the light zone (FIG. 12B), the TG mice treated with IN Suramin-Weekly showed a higher percentage of time spent in the Light Zone. All other treatment groups were comparable to the WT mice in the total time and percentage of time spent in the Light Zone (˜5-6 minutes and ˜50-60% of time).

### Light Zone Entries:

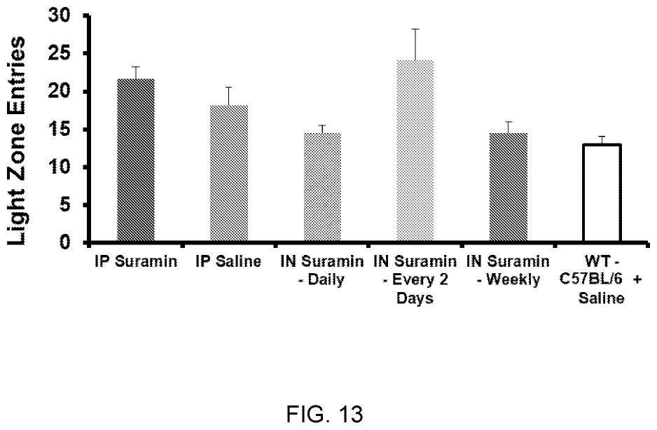

FIG. 13 shows the number of light zone entries. All the treatment groups showed a comparable or an increased number of Light Zone entries in comparison to the WT mice.

### Discussion:

### Dark Zone Entry Latency:

Naive, WT mice prefer darker areas over lighter areas of their environment. However, when presented with a novel environment, WT mice tend to explore. These two conflicting inclinations lead to observable anxiety-like symptoms. In assessing the dark zone entry latency in FIG. 11, the WT mice entered the dark box almost immediately (7.5 seconds) which is consistent with the WT mouse preference for dark environments, even in novel surroundings.

TG animals exhibited a latency in entering the dark zone and spent more time in the lighted area which may be due to a reduction in anxiety from the study drug treatments. Since all IN suramin groups showed comparable entry latencies (˜60-65 seconds), this would suggest that the frequency of dosing does not significantly affect anxiety-like responses in TG mice. However, the IN suramin-treated TG mice exhibited a dark zone entry latency that was almost double the latency of the IP suramin and IP saline groups (˜30-40 seconds) implying that the route of administration is having an impact on the results.

### Light Zone Time and Time Spent in Light Zone (%):

In anxiety models such as the light-dark test, WT mice spend less time in the light zone of the light/dark apparatus. However, WT mice treated with anxiolytic treatments typically exhibit an increase in the time spent in the light zone. In assessing the total time spent in the light zone (FIG. 12A), the TG mice treated with a variety of treatments all were observed to have a comparable total time spent in the light zone to the WT control group with three groups showing increased time in the Light Zone. The TG mice treated with IN Suramin (Weekly) showed a notable increased amount of time in the light zone (˜6.5 min).

Light Zone Entries: Assessment of Light Zone entries is an indirect way of measuring risk aversion as it relates to anxiety. Given the WT mice preference for dark enclosures, a mouse would “risk” subjecting itself to a less ideal/less comfortable setting by exiting the dark box and re-entering the light zone. In general, the TG treated mice exhibited a comparable or greater willingness to re-enter the light zone compared to the WT. Not only does this suggest a willingness to expose themselves to the light zone, but given the total time these two groups spent in the light zone was between 5-6 minutes, also shows a proclivity to explore the entire chamber (both the dark and light zones) equally.

### Conclusion:

In this study the TG animals treated with suramin exhibited a longer Dark Zone entry latency but a similar total time and percentage of time spent in the Light Zone, and Light Zone entry number to those observed in WT mice. The most substantial and significant effects are observed in the TG mice treated with IN Suramin (Weekly showing increased amount of time in the light zone. The observations in this study demonstrate that intranasal administration of suramin may lead to a reduction in anxiety-like behavior and a restoration of normal exploratory activity.

### Example 8: Evaluation of Suramin in a Locomotor Activity Test

### Objective

The purpose of this locomotor activity study was to test various suramin formulations and treatment routes and regimens in B6.129P2-Fmr1tm1Cgr/J transgenic (TG) mice to determine if there is an impact of these agents in locomotor activity, arousal, and willingness to explore compared to wild type mice and TG mice treated with IP saline as controls.

### Background:

The Locomotor Activity test is a means of establishing spontaneous locomotor activity, arousal, and willingness to explore in rodents. It is one of the most common rodent tests which can be used to test the effects of various medications on animal behavior in both wild type and genetically modified animals. See, Seibenhener M L, Wooten M C. Use of the Open Field Maze to measure locomotor and anxiety-like behavior in mice. J Vis Exp. 2015; (96):e52434. Published 2015 Feb. 6. doi: 10.3791/52434.

This study used a SmartCage™ system, which is an automated non-invasive rodent behavioral monitoring system which enables biomedical researchers to conduct a variety of neurobehavioral assays through consistent and accurate monitoring of rodent home cage activity and behavior. See, Xie et al, 2012, (Ibid.).

### Materials and Methods:

### Experimental Arms (Treatment Groups):

1. N=6 mice per group.
2. All behavioral testing included a group of untreated wild type controls.
3. Dosing for the various groups were as follows:
   a. Group 1: IP suramin, 20 mg/kg, administered weekly to animals beginning at 9 weeks of age and continuing for four weeks (i.e. given at Age Weeks 9, 10, 11 and 12).
   b. Group 2: IP saline, 5 mL/g, administered weekly to animals beginning at 9 weeks of age and continuing for four weeks (i.e. given at Age Weeks 9, 10, 11 and 12).
   c. Group 3: A formulation of IN suramin, at a concentration of 100 mg/mL×6 μL per spray, administered as one spray per nostril, one time per day, (interval of each application is around 2 min to ensure absorption) for 28 days (total of 56 sprays over 28 day period) beginning at 9 weeks of age (i.e. given daily during Age Weeks 9, 10, 11 and 12).
   d. Group 4: A formulation of IN suramin, at a concentration of 100 mg/mL×6 μL per spray, administered as one spray per nostril, one time every other day, for 28 days (total of 28 sprays over 28 day period) beginning at 9 weeks of age (i.e. given once every other day during Age Weeks 9, 10, 11 and 12).
   e. Group 5: A formulation of IN suramin, at a concentration of 100 mg/mL×6 μL per spray, administered as one spray per nostril, one time every week, for 4 weeks (28 days) (total of 8 sprays over 28 day period) beginning at 9 weeks of age (i.e. given once weekly during Age Weeks 9, 10, 11 and 12).
4. Behavioral tests for all groups (Groups 1-5) began the following day after the last day of IN dosing (Weeks 13-14 of age).
5. Blood samples (for PK testing) were collected from all mice at the end of Week 12 of age, just prior to starting the behavioral tests in Week 13 of age.
6. Brain tissue (for biochemistry testing) was harvested from all mice upon sacrifice at the conclusion of all behavioral testing at the end of Week 13-14 of age.

### Animals:

Male B6.129P2-Fmr1tm1Cgr/J TG (TG) mice were purchased from Jackson Laboratories, Bar Harbor, Maine. These mice were of approximately 8(+1) weeks of age. Mice were maintained in group cages (6 mice per cage based on treatment group) in a controlled environment (temperature: 21.5±4.5° C./relative humidity: 35-55%) under a standard 12 hour light/12 hour dark lighting cycle (lights on at 06:00). Mice accommodated to the research facility for the remainder of the week. Dosing began on the following Monday. Body weights of all mice were recorded for health monitoring purposes.

### Experimental Methods:

Dosing was carried out over the course of 28 days as instructed by the study Sponsor. The second behavioral test, SmartCage™ Locomotion Monitoring, was performed from the beginning of the 12 hour dark cycle (˜5:00 PM) on the first day until the morning of the second day, in the first 32 mice, and from the beginning of the 12 hour dark cycle on the second day to the morning of the third day, for the second 32 mice.
1. Day 1 to Day 2: Locomotor Activity recording is assessed using the SmartCage™
2. B6.129P2-Fmr1tm1Cgr/J TG Mice received IP injections and IN dosing approximately 30 min prior to being placed in the SmartCage™
3. Mice were placed in the SmartCage™ at 4:00 PM (1 hour prior to the beginning of the dark phase of the 12 h:12 h dark/light cycle).
4. An ˜24 h Locomotion recording of the mice freely moving in the SmartCage™ was taken.
5. The first 12 hours on each graph (the grey-shaded box) represents the dark phase of the dark/light cycle.
6. Wildtype (WT) Control data (collected separately prior to the beginning of the dosing of the dosing of the B6.129P2-Fmr1tm1Cgr/J TG mice) was added to the final data analysis to serve as a comparison for naive, male C57BL/6 mice.

Each mouse was placed in a clean plastic, transparent home cage within the SmartCage™. Each home cage consisted of a thin layer of bedding (Sani Chips, 7090A; Envigo). Rodent chow (Teklab Diet 2018, Envigo) and water gel (Hydrogel, Teklab) were placed directly in the home cage. The mice roamed freely within their home cage for the entire duration of the SmartCage™ locomotion recording (˜24 hours). Active Time, Travel Distance, and Rearing Activity were assessed for each mouse. Data analyzed based on treatment group.

### Results:

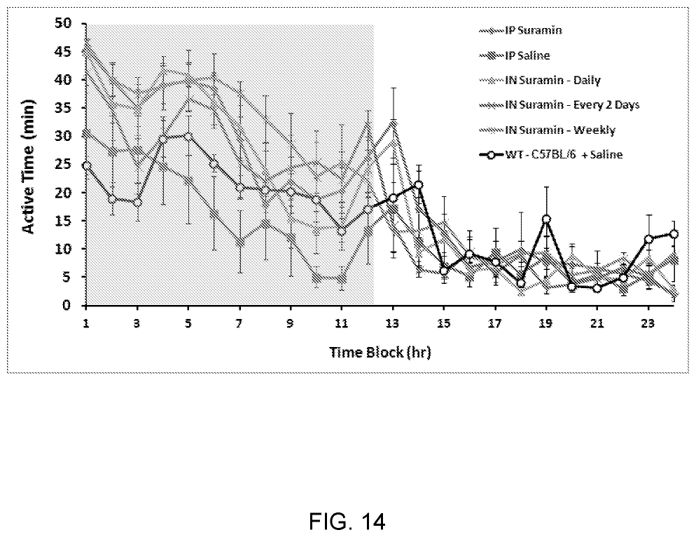

Active Time: FIG. 14 shows the mouse active time in minutes per hour time block. The mice from all treatment groups display higher activity during the dark cycle and lower activity levels during the light cycle.

All IN Suramin (Daily, Weekly, and Every 2 Days) treatment groups displayed greater activity compared to the WT control group. In contrast, the IP Saline group shows comparable, and in some time blocks, lower active time than the WT control group.

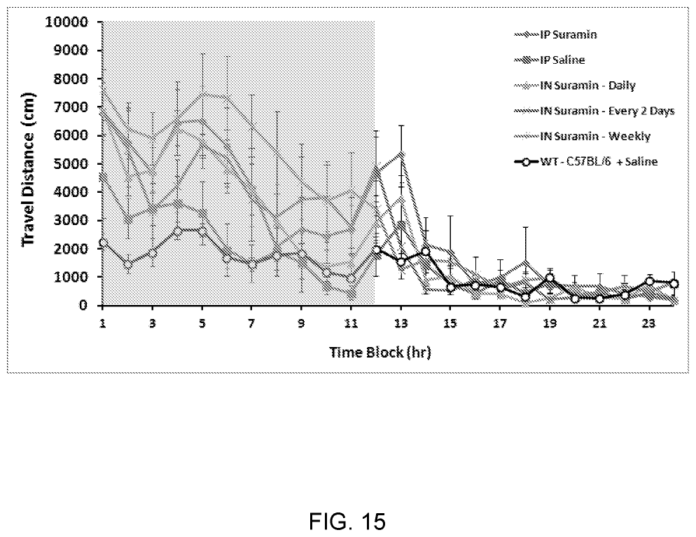

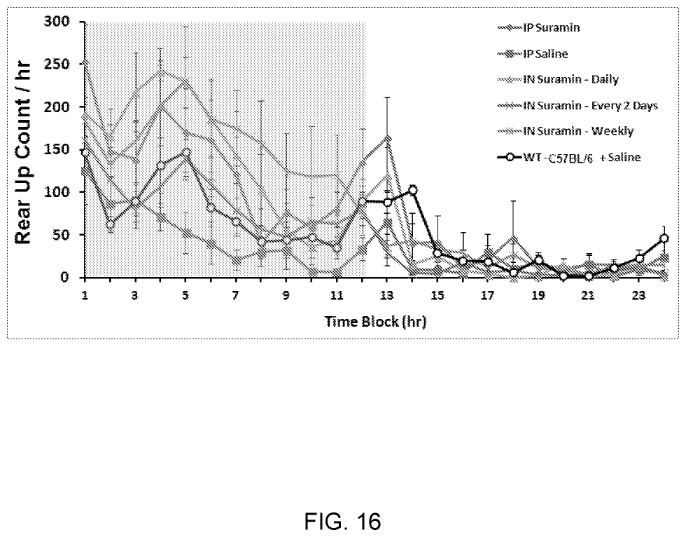

Travel Distance: FIG. 15 shows the travel distance in centimeters plotted per hour time block. The mice from all drug-treated groups displayed significantly greater distances traveled than the WT and IP Saline control groups. This finding was particularly pronounced during the dark period and less consistent during the light period.
Rearing Count: FIG. 16 shows the rearing count per hour time block. The drug-treated mice from the IN- and IP-administered drug treatment groups display greater and more frequent rearing activity than the WT control group and the IP saline group. The TG mice treated with IN Suramin every 2 days displayed rearing activity that was comparable to the WT control group.

### Discussion:

Active Time: Active time quantifies how much time the mice are active including time spent walking/running, rearing, and/or rotating. The mice from all treatment groups display higher activity during the dark cycle and minimal activity during the light cycle (FIG. 14) as is consistent with their nature pattern of activity. This was expected given that mice are nocturnal rodents. Since the monitoring started at the initiation of the dark cycle, most activity occurred in the first 12 hours of the locomotion recording. In general, dosing via various routes of administration can impact locomotor activity if performed shortly before the beginning of the locomotion recording. An early spike in activity can result from hyperactivity due to the recent injection whereas a sudden decrease in activity can be due to physical impairment of the mouse if the route of drug administration causes physical discomfort.
All IN Suramin (Daily, Weekly, and Every 2 Days) treatment groups displayed greater activity compared to the WT control group. In contrast, the IP Saline group shows comparable, if not slightly lesser, activity than the WT control group. Taken together, these results suggest that the actual dosing itself did not directly cause changes in locomotor activity. The results suggest that the active treatment interventions are leading to an increased locomotor response.
Travel Distance: Travel distance quantifies the total distance in cm mice cover on the x- and y-axes while roaming and exploring their respective home cage. The mice from all drug-treated groups displayed significantly greater distances traveled than the WT and IP Saline control groups (FIG. 15). Given that mice from all drug-treated groups, regardless of route of drug administration, displayed increased travel distance, this suggests that none of the drug treatments impaired locomotor activity or increased anxiety. In contrast, the drug-treated mice showed an increased willingness to explore their home cage. This finding is consistent with the signs of reduced anxiety that were observed in the Light/Dark Test (see report of Light/Dark Test of Anxiety Like Behavior).
Rearing Count: Rearing activity measures the number of times a mouse extends upward from its hindlimbs to reach towards the top of its home cage. Rearing activity is measured by the IR sensors on the Z-axis of the SmartCage™. Given that both food and water (hydrogel) were placed directly on the floor of each mouse's home cage, there is no need for the mice to reach up for food and/or water. Therefore, the Rear Up Count measured by the SmartCage™ serves as an indication of the mouse's general activity and exploratory behavior. As observed in the Active Time and Travel Distance graphs, the drug-treated mice from the IN- and IP-administered groups generally displayed more frequent rearing activity than the WT control group and the IP saline group. When these results are combined with the Active Time and Travel Distance data, the Rearing Activity data is consistent in showing increased activity, arousal and willingness to explore in the all Suramin-treated groups.

### Conclusion:

Taken together, the findings from this locomotor activity suggest that the Male B6.129P2-Fmr1tm1Cgr/J TG mice treated with study medications displayed increased activity and a greater willingness to explore their environment compared with TG mice treated with IP saline or the Wild Type mice. When looking at the locomotor activity data combined with the light/dark data, it is reasonable to suggest that treatment with anti-purinergic medications led to greater exploration of the animals' environment in the dark phase, potentially due to reduced anxiety. None of the treatments changed the inactive state of the mice (or total sleep) during light phase.

### Example 9: Evaluation of Suramin in a Social Interaction Study

### Objective

The purpose of this social interaction activity study was to test to test various suramin formulations and treatment routes and regimens in B6.129P2-Fmr1tm1Cgr/J transgenic (TG) mice to determine if there is an impact of these agents on social behavior compared to wild type mice and TG mice treated with IP saline as controls.

### Background:

Social interactions are a fundamental and adaptive component of the biology of numerous species including mice and rats. Social recognition is critical for the structure and stability of the networks and relationships that define societies. A variety of neuropsychiatric disorders are characterized by disruptions in social behavior and social recognition, including depression, autism spectrum disorders, bipolar disorders, obsessive-compulsive disorders, and schizophrenia.

The mouse social interaction study employed a three-chamber paradigm test known as Crawley's sociability and preference for social novelty protocol has been successfully employed to study social affiliation and social memory in several inbred and mutant mouse lines. The test is based on the principal of free choice by a subject mouse to spend time in any of three box's compartments during two experimental sessions, including indirect contact with one or two mice with which it is unfamiliar. To quantitate social tendencies of the experimental mouse, the main tasks are to measure a) the time spent with a novel mouse and b) preference for a novel vs. a familiar mouse. Thus, the experimental design of this test allows evaluation of two critical but distinguishable aspects of social behavior: social affiliation/motivation, as well as social memory and novelty. “Sociability” in this case is defined as propensity to spend time with another mouse, as compared to time spent alone in an identical but empty chamber. See, Moy S S, Nadler J J, Perez A, Barbaro R P, Johns J M, Magnuson T R, Piven J, Crawley J N. Sociability and preference for social novelty in five inbred strains: an approach to assess autistic-like behavior in mice. Genes, brain, and behavior. 2004; 3:287-302; and Kaidanovich-Beilin O, Lipina T V, Takao K, Eede Mvan, Hattori S, Laliberte C, Khan M, Okamoto K, Chambers J W, Fletcher P J, Macaulay K, Doble B W, Henkelman M, Miyakawa T, Roder J, Woodgett J R. Abnormalities in brain structure and behavior in GSK-3alpha mutant mice. Molecular brain. 2009; 2:35-35.

This study used a SmartCage™ system, which is an automated non-invasive rodent behavioral monitoring system which enables biomedical researchers to conduct a variety of neurobehavioral assays through consistent and accurate monitoring of rodent home cage activity and behavior. See, Xie et al, 2012 (Ibid.).

### Materials and Methods:

### Experimental Arms (Treatment Groups):

1. N=6 mice per group.
2. All behavioral testing included a group of untreated wild type controls.
3. Dosing for the various groups were as follows:
   a. Group 1: IP suramin, 20 mg/kg, administered weekly to animals beginning at 9 weeks of age and continuing for four weeks (i.e. given at Age Weeks 9, 10, 11 and 12).
   b. Group 2: IP saline, 5 mL/g, administered weekly to animals beginning at 9 weeks of age and continuing for four weeks (i.e. given at Age Weeks 9, 10, 11 and 12).
   c. Group 3: A formulation of IN suramin, at a concentration of 100 mg/mL×6 μL per spray, administered as one spray per nostril, one time per day, (interval of each application is around 2 min to ensure absorption) for 28 days (total of 56 sprays over 28 day period) beginning at 9 weeks of age (i.e. given daily during Age Weeks 9, 10, 11 and 12).
   d. Group 4: A formulation of IN suramin, at a concentration of 100 mg/mL×6 μL per spray, administered as one spray per nostril, one time every other day, for 28 days (total of 28 sprays over 28 day period) beginning at 9 weeks of age (i.e. given once every other day during Age Weeks 9, 10, 11 and 12).
   e. Group 5: A formulation of IN suramin, at a concentration of 100 mg/mL×6 μL per spray, administered as one spray per nostril, one time every week, for 4 weeks (28 days) (total of 8 sprays over 28 day period) beginning at 9 weeks of age (i.e. given once weekly during Age Weeks 9, 10, 11 and 12).
4. Behavioral tests for all groups (Groups 1-5) began the following day after the last day of IN dosing (Weeks 13-14 of age).
5. Blood samples (for PK testing) were collected from all mice at the end of Week 12 of age, just prior to starting the behavioral tests in Week 13 of age.
6. Brain tissue (for biochemistry testing) was harvested from all mice upon sacrifice at the conclusion of all behavioral testing at the end of Week 13-14 of age.

### Animals:

Male B6.129P2-Fmr1tm1Cgr/J TG (TG) mice were purchased from Jackson Laboratories, Bar Harbor, Maine. These mice were of approximately 8 (±1) weeks of age. Mice were maintained in group cages (6 mice per cage based on treatment group) in a controlled environment (temperature: 21.5±4.5° C./relative humidity: 35-55%) under a standard 12 hour light/12 hour dark lighting cycle (lights on at 06:00). Mice accommodated to the research facility for the remainder of the week. Dosing began on the following Monday. Body weights of all mice were recorded for health monitoring purposes.

### Experimental Methods:

Dosing was carried out over the course of 28 days as instructed by the study Sponsor. The third behavioral test, the Social Interaction Test, was performed and completed 4 days later. Each Subject Mouse was paired with Stranger Mice from different home cage. This ensured that each Subject Mouse did not have any prior interactions with the Stranger Mice, thus minimizing potential biases.
1. Social Interaction using the SmartCage™—Attach the social interaction rodent compartments on the far ends of the mouse SmartCage™ chamber.
2. Habituation—For minutes 0-5, the subject mouse could roam the chamber freely while both social interaction compartments remain empty.
3. Mice that show a heavy preference for either zone (zone 1—Stranger 1 compartment; zone 3—Stranger 2 compartment) should be discarded; mice that show a ˜50:50 exploration demonstrate unbiased exploration.
4. Sociability—from minutes 5-10, place Stranger 1 mouse in the Stranger 1 compartment in Zone 1; allow the subject mouse to explore freely.
5. Social Novelty—from minutes 10-15, place Stranger 2 mouse in the Stranger 2 compartment in Zone 4; allow the mouse to explore freely.
6. Occupancy time in Zone 1 and Zone 3 is analyzed and used to assess how much preference, if at all, the subject mouse has for either Stranger mice.
7. Ideally, Stranger mice should be of similar age/weight and gender as the Subject mouse, but NOT from the same home cage.
8. Wildtype Control data (collected separately prior to the beginning of the dosing of the dosing of the B6.129P2-Fmr1tm1Cgr/J TG mice) was added to the final data analysis to serve as a comparison for naive, male C57BL/6 mice.

The entire Social Interaction Chamber as well as the Stranger compartments were wiped down thoroughly with a light towel doused in 0.05% bleach in between each Social Interaction test. This reduced the potential for smells from prior trials influencing the behavior of the test subjects. In addition, Social Interaction tests were performed under fluorescent ceiling lights which provided equal lighting over the entire Social Interaction Chamber including both Social Interaction compartments.

### Results:

### Habituation:

In the first five minutes of the Social Interaction test (Habituation Phase), each subject mouse can freely explore the Social Interaction chamber. At this point, the two “Stranger” compartments are empty. In general, a naive, WT mouse would explore and investigate both Stranger Compartments equally. The SmartCage™'s assessment of Occupancy Time gives a direct measurement of how much time each subject mouse spends exploring and investigating each Stranger Compartment.

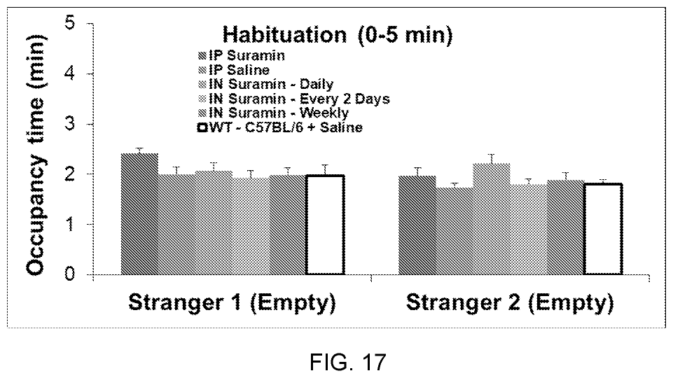

FIG. 17 show Habituation for minutes 0-5 and the occupancy time (minutes) for each stranger compartment. All drug treatment groups, as well as the WT Control group, showed equal time spent exploring the two Stranger Compartments. This Habituation Phase ensured that the WT control mice as well as the TG mice showed no inherent bias to either side of the Social Interaction Chamber prior to introduction of the Stranger Mice.

The activity level and occupancy time for each of the TG treatment groups for each compartment is comparable to that observed from the WT Control group.

### Sociability:

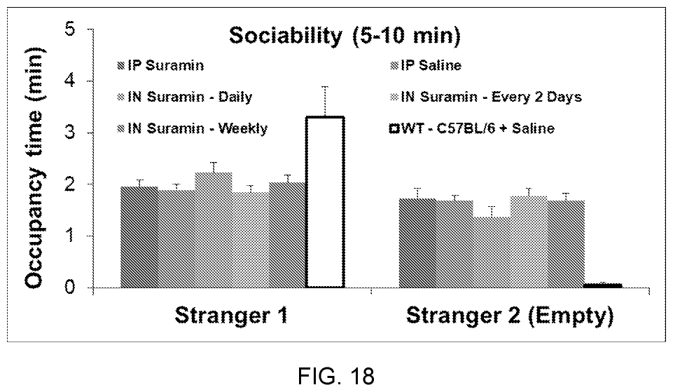

FIG. 18 shows the Sociability Analysis (minutes 5-10) depicting occupancy time in minutes for each treatment group for Stranger compartments 1 and 2. With the introduction of the Stranger 1 mouse, the focus of the subject mouse turns to the Stranger 1 compartment. The occupancy time is greater in the Stranger 1 compartment than the occupancy time in the Stranger 2 compartment. The WT mice spend more time in the Stranger 1 compartment than the TG mice. Notably, the TG mice all show greater occupancy time in Stranger compartment 2 than the WT mice, even though it is empty.

### Social Novelty:

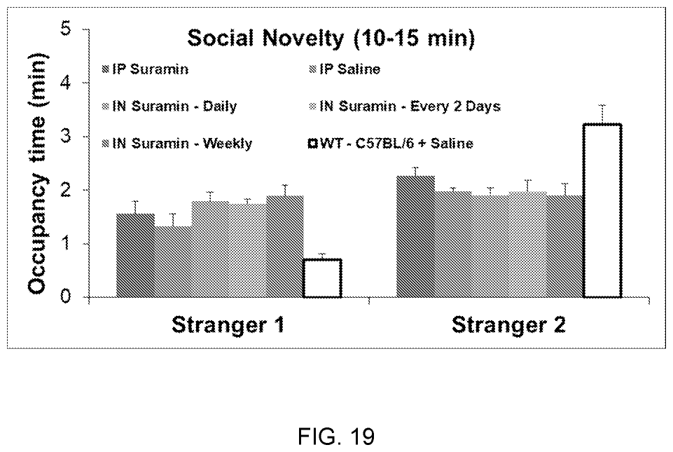

FIG. 19 shows Social Novelty with occupancy time (minutes) measured in each compartment after the introduction of a new mouse in Stranger compartment 2. With the introduction of the Stranger 2 mouse, the focus of the subject mouse turns to the Stranger 2 compartment. The mice from all drug-treated TG groups as well as the WT Control group show a greater occupancy time in the Stranger 2 compartment during the Social Novelty phase (minutes 10-15) with the WT mice showing the greatest occupancy time.

### Discussion:

As evidenced by the by the Habituation Phase of the Social Interaction Test, all TG mice drug treatment groups, as well as the WT control group, habituated well and showed an equal amount of time spent exploring the two Stranger compartments.

In the Sociability stage, when compared to the WT mice, the TG mice treated with study drug spent less time with the Stranger 1 mouse and did not establish definitive sociability.

Likewise, when the Stranger 2 mouse was introduced, these same groups all significantly shifted their attention to the Stranger 2 compartment. However, the WT mouse still showed the greatest occupancy time with the Stranger 2 mouse indicating a higher level of sociability compared with the TG mice treated with study drug. A possible explanation for this difference may be that the WT mice were not subject to the same dosing paradigm nor were they housed with their cage mates for as long as the TG mice were. Therefore, the WT mice may have displayed greater sociability due to the originality of social interaction.

TG mice typically show less sociability compared with WT mice. As observed in the light/dark test data and locomotor activity test data, the medication treated TG mice showed reduced anxiety and a tendency to explore which may lead to enhanced sociability. This test also requires intact short-term memory as the mouse must recall that they have previously socialized with the Stranger 1 mouse when the Stranger 2 mouse is introduced. The intact short-term memory allows for social novelty/social differentiation. These findings suggest that drug treated TG mice showed improved sociability and intact short-term memory, although not a full restoration of these abilities as observed in the WT mice

### Conclusion:

Consistent with other behavioral assessments in this study, the B6.129P2-Fmr1tm1Cgr/J TG mice show deficits in sociability and response to social novelty. The findings from this Social Interaction Study suggest that the B6.129P2-Fmr1tm1Cgr/J TG mice treated with study medications exhibited increased social activity, reduced anxiety, and a greater willingness to explore their environment. Taken together, it also suggests that anti-purinergic receptor medications may restore normal short-term memory and social activity that are typically absent in this TG mouse model.

### Example 10: Evaluation of Suramin in a Spatial Learning and Memory Study

### Objective

The purpose of the Morris Water Maze study was to test to test various suramin formulations and treatment routes and regimens in B6.129P2-Fmr1tm1Cgr/J transgenic (TG) mice to determine if there is an impact of these agents on spatial learning and memory compared to wild type mice and TG mice treated with IP saline as controls.

### Background:

The Morris Water Maze Test (MWM) is one of the most widely used tasks in behavioral neuroscience for studying the psychological processes and neural mechanisms of spatial learning and memory. MWM is a rodent test of spatial learning that relies on distal cues to navigate from a starting point around the perimeter of an open swimming arena to locate a submerged escape platform. Spatial learning is assessed across repeated trials and reference memory is determined by preference for the platform area when the platform is absent. Spatial memory is assessed during a probe trial in which the platform is removed and the percentage of time the animals spend searching in the spatial location where the platform was previously positioned (target quadrant) is measured. Spatial learning in humans is a form of declarative memory. Several studies have used computer systems with virtual mazes and navigational tasks to assess human spatial learning and memory.

The MWM has proven to be a robust and reliable test that is strongly correlated with hippocampal synaptic plasticity and NMDA receptor function. See, Vorhees, C., Williams, M. Morris water maze: procedures for assessing spatial and related forms of learning and memory. Nat Protoc 1, 848-858 (2006).

This study used a SmartCage™ system, which is an automated non-invasive rodent behavioral monitoring system which enables biomedical researchers to conduct a variety of neurobehavioral assays through consistent and accurate monitoring of rodent home cage activity and behavior. See, Xie et al, 2012 (Ibid.).

### Materials and Methods:

### Experimental Arms (Treatment Groups):

1. N=6 mice per group.
2. All behavioral testing included a group of untreated wild type (WT) controls.
3. Dosing for the various groups were as follows:
   a. Group 1: IP suramin, 20 mg/kg, administered weekly to animals beginning at 9 weeks of age and continuing for four weeks (i.e. given at Age Weeks 9, 10, 11 and 12).
   b. Group 2: IP saline, 5 mL/g, administered weekly to animals beginning at 9 weeks of age and continuing for four weeks (i.e. given at Age Weeks 9, 10, 11 and 12).
   c. Group 3: A formulation of IN suramin, at a concentration of 100 mg/mL×6 μL per spray, administered as one spray per nostril, one time per day, (interval of each application is around 2 min to ensure absorption) for 28 days (total of 56 sprays over 28 day period) beginning at 9 weeks of age (i.e. given daily during Age Weeks 9, 10, 11 and 12).
   d. Group 4: A formulation of IN suramin, at a concentration of 100 mg/mL×6 μL per spray, administered as one spray per nostril, one time every other day, for 28 days (total of 28 sprays over 28 day period) beginning at 9 weeks of age (i.e. given once every other day during Age Weeks 9, 10, 11 and 12).
   e. Group 5: A formulation of IN suramin, at a concentration of 100 mg/mL×6 μL per spray, administered as one spray per nostril, one time every week, for 4 weeks (28 days) (total of 8 sprays over 28 day period) beginning at 9 weeks of age (i.e. given once weekly during Age Weeks 9, 10, 11 and 12).
4. Behavioral tests for all groups (Groups 1-5) began the following day after the last day of IN dosing (Weeks 13-14 of age).
5. Blood samples (for PK testing) were collected from all mice at the end of Week 12 of age, just prior to starting the behavioral tests in Week 13 of age.
6. Brain tissue (for biochemistry testing) was harvested from all mice upon sacrifice at the conclusion of all behavioral testing at the end of Week 13-14 of age.

### Animals:

Male B6.129P2-Fmr1tm1Cgr/J TG (TG) mice were purchased from Jackson Laboratories, Bar Harbor, Maine. These mice were of approximately 8 (±1) weeks of age. Mice were maintained in group cages (6 mice per cage based on treatment group) in a controlled environment (temperature: 21.5±4.5° C./relative humidity: 35-55%) under a standard 12 hour light/12 hour dark lighting cycle (lights on at 06:00). Mice accommodated to the research facility for the remainder of the week. Dosing began on the following Monday. Body weights of all mice were recorded for health monitoring purposes.

### Experimental Methods:

Dosing was carried out over the course of 28 days as instructed by the study Sponsor. The fourth behavioral test, the Morris Water Maze, was performed over five days from days 33 through 37. For the first four days, each mouse was subject to four trials in the Morris Water Maze. In each trial, each mouse was given 60 seconds to locate and situate itself on the target platform. If the mouse did not locate the target platform after 60 seconds, the tester manually placed the mouse on the platform and allowed the mouse to sit atop the platform for at least 20 seconds. On Day 5, each mouse was subject to two trial runs before the probe test. After completing the two Day trial runs, the target platform is removed from the Morris Water Maze tank. In the probe test, each mouse is released into the water tank and allowed to roam the tank freely. The amount of time spent in the zone where the target platform was originally situated was recorded for each mouse.
1. Morris Water Maze: Water pool diameter is 105 cm; Water is colored with washable white paint; Water temperature is 21-22 degrees centigrade; The platform is 8.5 cm×13.5 cm size and located at 1.5 cm deep under the water.
2. Prior to the beginning of the Water Maze training, each mouse is placed on top of the platform for 20 seconds.
3. The mouse is released from different location of the tank to find the platform; The latency for mice to reach the platform are automatically recorded using ANY-Maze Behavior Tracking Software.
4. The acquisition: 5 day's training. The first 4 days is 4 trials per day and day 5 training is 2 trials before probe test. The mouse is placed on platform for 20 seconds if the animal is unable to find the platform.
5. After reaching the platform, the mouse is immediately removed from the platform and returned to its home cage, thus completing the acquisition training.
6. Probe test: After training, the mouse is released from different location of the water tank to find the original platform which is removed from the water.
7. The mouse may freely explore the platform for 1 minutes, and the time spent in the target quadrant is recorded by Any-Maze Behavioral Tracking Software.
8. Spatial learning and memory behavior are assessed based on the software monitoring of time spent in the target quadrant, the number of the target quadrant entries, and % Time Spent in the target quadrant.

Data is grouped together based on treatment group (Wild type, IP Suramin, IP Saline, IN Suramin-Daily, IN Suramin-Every Other Day, and IN Suramin-Weekly). The Morris Water Maze tank was filled with water that was dyed a milky-white hue. This allowed for the MWM software to track the black-colored mice with higher resolution while providing greater contrast between the mice and the water in the video playback. After each trial, each mouse was manually removed from the water and lightly dried by the tester using a soft towel, hand-drying technique. The subject mouse was then placed under a heating lamp to ensure drying while also reducing the likelihood of hypothermia. No mice showed any adverse reaction from the daily, multiple trial runs.

The MWM test was conducted in two phases: acquisition and probe. In the acquisition phase, reference memory protocols were used in which the platform is in a fixed location relative to the room cues across days. The animals are placed into the water at and facing the sidewalls of the pool and at different starting positions across trials. They quickly learn to swim to the correct location with decreasing escape latencies and with a more direct swim path.

The tracking system measures the gradually reduced escape latency across trials and parameters such as path-length, swim-speed, and directionality in relation to platform location. Observation of the animals reveals that, having climbed onto the escape platform, they often rear up and look around, as if trying to identify their location in space. Rearing habituates over trials, but then dishabituates if the hidden platform is moved to a new location or removed entirely (as in the Probe test).

During or after training is complete, the experimenter conducts a probe test in which the escape platform is removed from the pool and the animal is allowed to swim for 60 sec. Typically, a well-trained mouse will swim to the target quadrant of the pool and then swim repeatedly across the former location of the platform before starting to search elsewhere. This spatial bias, measured in various ways, constitutes evidence for spatial memory. Mice with lesions of the hippocampus and dentate gyrus, subiculum, or combined lesions, do poorly in post-training probe tests.

### Results:

### Acquisition Test

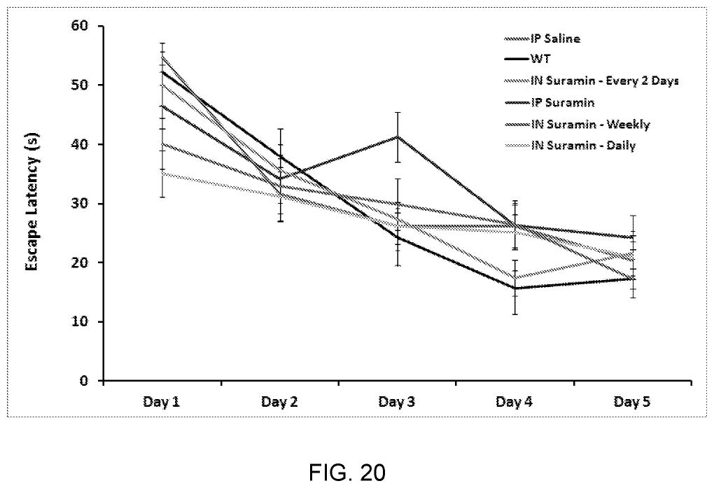

FIG. 20 shows the Acquisition Test escape latency (seconds) for each of the treatment groups on days 1-5. All mice showed a decreased escape latency from days 1 through 5, thus exhibiting a consistent but gradual learning of the spatial parameters of the Morris Water Maze tank. All TG mice showed a comparable spatial awareness acquisition process to the WT mice, regardless of treatment group. The WT control group required 17 seconds to acquire the target platform. All other TG mice treatment groups located the platform within 20-27 seconds by the final day of training.

### Probe Test:

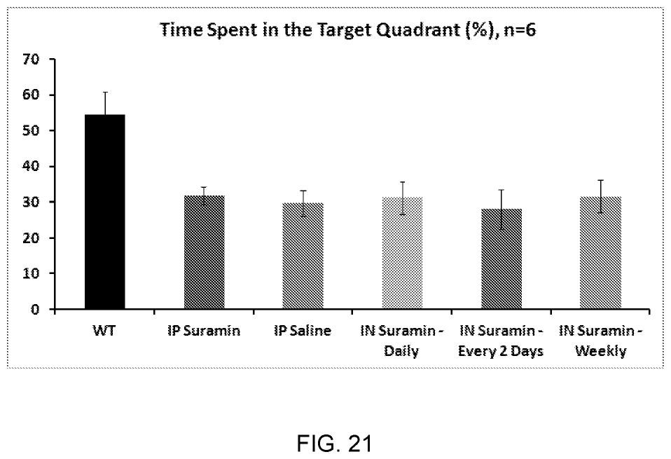

FIG. 21 from the Probe Test shows the time (seconds) spent in the target quadrant attempting to locate the escape platform. The WT Control group displayed the longest occupancy time in the target quadrant at approximately 52.53%. All TG mice spent significantly less time in the target quadrant than the WT Control group. All TG mice treated with some form of Suramin spent between 28%-32% of their probe trial time in the target quadrant.

### Discussion:

Acquisition Phase: The WT and TG mice, regardless of treatment group, displayed comparable learning, spatial awareness, and spatial recognition.
Probe Phase: None of the treated TG mice spent as much time in the Target Quadrant as the WT mice.

### Conclusion:

The WT and treated TG mice showed a steady decrease in escape latency over time exhibiting a consistent but gradual learning of the spatial parameters of the Morris Water Maze tank. The overall occupancy time in the target quadrant was greater in WT compared with TG mice during the probe phase. This suggests that the study treatments in the TG mice did not have any negative or debilitating effect on normal cognitive function or spatial learning and memory.

### Example 11: Evaluation of Suramin in a Contextual Conditioning Memory Test for Learning and Memory

### Objective

The purpose of the Step Through Passive Avoidance Test was to test to test various suramin formulations and treatment routes and regimens in B6.129P2-Fmr1tm1Cgr/J transgenic (TG) mice to determine if there is an impact of these agents on learning and memory compared to wild type mice and TG mice treated with IP saline as controls.

### Background:

The Passive Avoidance task is useful for evaluating the effect of novel chemical entities on learning and memory as well as studying the mechanisms involved in rodent models of CNS disorders. In this test, the test chamber is divided into a lighted compartment and a dark compartment, with a gate between the two. The test animals explored both compartments on the first day. The next day, they are given a mild foot shock in the dark compartment and they will learn to associate the dark compartment with the foot shock. To test their learning and memory, the mice are then placed back in the lighted compartment. Passive avoidance behavior of rodents is defined as the suppression of their innate preference for the dark compartment. Mice with normal learning and memory will avoid entering the dark chamber. Learning and memory from the previous day is measured by recording the latency to cross through the gate between the two compartments. See, J. David Sweatt, Chapter 4: Rodent Behavioral Learning and Memory Models, Editor: J. David Sweatt. Mechanisms of Memory (Second Edition), Academic Press, 2010, Pages 76-103, ISBN 9780123749512.

This study used a SmartCage™ system, which is an automated non-invasive rodent behavioral monitoring system which enables biomedical researchers to conduct a variety of neurobehavioral assays through consistent and accurate monitoring of rodent home cage activity and behavior. See, Xie et al, 2012.

### Materials and Methods:

### Experimental Arms (Treatment Groups):

1. N=6 mice per group.
2. All behavioral testing included a group of untreated wild type (WT) controls.
3. Dosing for the various groups were as follows:
   a. Group 1: IP suramin, 20 mg/kg, administered weekly to animals beginning at 9 weeks of age and continuing for four weeks (i.e. given at Age Weeks 9, 10, 11 and 12).
   b. Group 2: IP saline, 5 mL/g, administered weekly to animals beginning at 9 weeks of age and continuing for four weeks (i.e. given at Age Weeks 9, 10, 11 and 12).
   c. Group 3: A formulation of IN suramin, at a concentration of 100 mg/mL×6 μL per spray, administered as one spray per nostril, one time per day, (interval of each application is around 2 min to ensure absorption) for 28 days (total of 56 sprays over 28 day period) beginning at 9 weeks of age (i.e. given daily during Age Weeks 9, 10, 11 and 12).
   d. Group 4: A formulation of IN suramin, at a concentration of 100 mg/mL×6 μL per spray, administered as one spray per nostril, one time every other day, for 28 days (total of 28 sprays over 28 day period) beginning at 9 weeks of age (i.e. given once every other day during Age Weeks 9, 10, 11 and 12).
   e. Group 5: A formulation of IN suramin, at a concentration of 100 mg/mL×6 μL per spray, administered as one spray per nostril, one time every week, for 4 weeks (28 days) (total of 8 sprays over 28 day period) beginning at 9 weeks of age (i.e. given once weekly during Age Weeks 9, 10, 11 and 12).
4. Behavioral tests for all groups (Groups 1-5) began the following day after the last day of IN dosing (Weeks 13-14 of age).
5. Blood samples (for PK testing) were collected from all mice at the end of Week 12 of age, just prior to starting the behavioral tests in Week 13 of age.
6. Brain tissue (for biochemistry testing) was harvested from all mice upon sacrifice at the conclusion of all behavioral testing at the end of Week 13-14 of age.

### Animals:

Male B6.129P2-Fmr1tm1Cgr/J TG (TG) mice were purchased from Jackson Laboratories, Bar Harbor, Maine. These mice were of approximately 8 (±1) weeks of age. Mice were maintained in group cages (6 mice per cage based on treatment group) in a controlled environment (temperature: 21.5±4.5° C./relative humidity: 35-55%) under a standard 12 hour light/12 hour dark lighting cycle (lights on at 06:00). Mice accommodated to the research facility for the remainder of the week. Dosing began on the following Monday. Body weights of all mice were recorded for health monitoring purposes.

### Experimental Methods:

Dosing was carried out over the course of 28 days as instructed by the study Sponsor. The fifth and final behavioral test, the Step Through (ST) Passive Avoidance Test, was performed over two days (days 38 and 39) with the first day as a Training Day and the second day as a Test Day. Passive avoidance is fear-motivated tests classically used to assess short-term or long-term memory on rodents. The Passive avoidance paradigm requires the subjects to behave contrary to their innate tendencies for preference of dark areas and avoidance of bright ones. The dark box (red transparent enclosure with an opening for the mouse to enter) used in the ST test is the same dark box used in the “Light-Dark” test; in the ST test, the dark box is placed atop the metal foot shock grid which sends an electric shock to the mouse as soon as the mouse enters the dark box. Each mouse was trained individually, one at a time. As soon as the mouse entered the dark box, the mouse received a direct electric shock to its hind paws. After receiving the foot shock, the experimenter manually removed the mouse from the SmartCage™ and returned it to its home cage. In between each training session, the metal grid and dark box were gently wiped down with 0.05% bleach. This minimized any potential biases that may have occurred due to residual odors or debris (hair, bedding, food particles) from prior training sessions.
1. Prior to the beginning of the Step-Through (ST) training, a dark box (red transparent enclosure with an opening for the mouse to enter) is placed on top of the metal foot shock grid within the SmartCage™
2. An individual mouse is placed in the open/non-dark box side (“Light Zone”), with its head facing away from the dark box.
3. The mouse is then allowed to freely explore the SmartCage™ and enter the dark box “Dark Zone” at its own discretion; dark box entry latency is automatically recorded.
4. As soon as the mouse enters the dark box and is situated in the dark enclosure for at least 1 second, the rat receives a foot shock (via the metal grid) that lasts for 2 seconds.
5. After receiving the foot shock, the mouse is immediately removed from the SmartCage™ and returned to its home cage, thus completing the Step-Through Training.
6. 24 h post-ST training, the mouse is placed in the same SmartCage™ setup from the previous day; the mouse is placed in the open/non-dark box side.
7. The mouse may freely explore the SmartCage™ for 5 minutes, and avoidance of the Dark Box due to contextual-fear association is assessed.
8. Contextual Fear-conditioned behavior is assessed based on the SmartCage™ monitoring of time spent in the Light Zone, the number of Light Zone entries, and % Time Spent in the Light Zone.
9. In addition, a comparison of dark box entry latency between the training day (pre-foot shock) and 24 h post-training (Test Day) is performed.
10. Data is grouped together based on treatment group (IP Suramin, IP Saline, IN Suramin-Daily, IN Suramin-Every Other Day, IN Suramin-Weekly).

### Results:

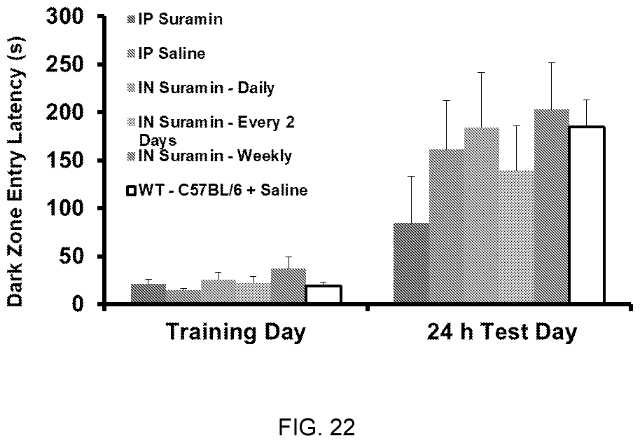

FIG. 22 shows the Dark Zone Entry Latency (seconds) for the training day and for the test day 24 hours later for each treatment group. The latency for each mouse to enter the dark box was compared between the Training Day and the Test Day (24 h post-foot shock). All treatment groups entered the dark compartment in less than 50 seconds on the Training Day. On the Test Day, 24 hours later, all treatment groups retained memory of the mild foot shock and avoided entering the dark compartment for a longer time than on the Training Day. The IP suramin group had the shortest latency for entering the dark compartment and all other treatment groups had a longer latency which was similar to that observed in the WT mice.

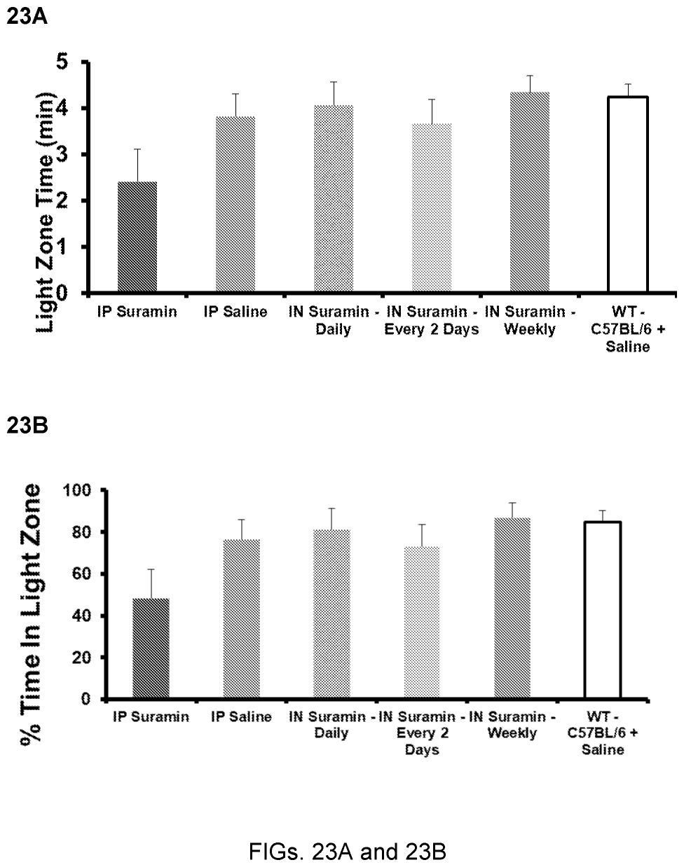

FIG. 23A shows the total light zone time (minutes) and FIG. 23B shows the percentage of time spent in the light zone on the test day. The WT and TG mice treated with IN suramin and IN saline show a similar total time spent in the light zone and greater than 70% of time spent in the light zone. The IP Suramin showed a lower total time and approximately 50% of their time in the light zone.

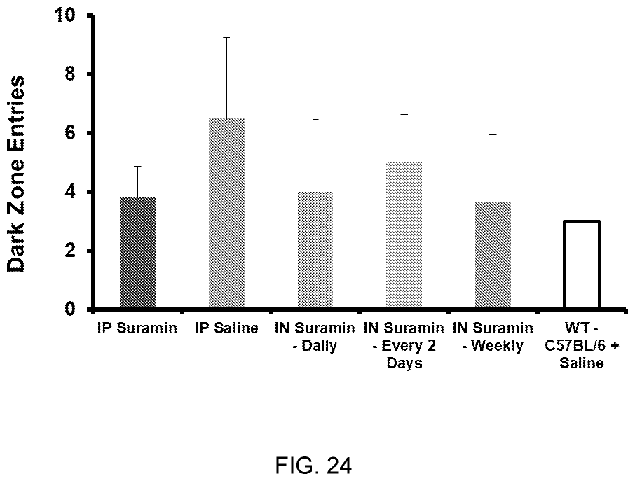

FIG. 24 shows the total number of Dark Zone Entries per treatment group. The IP saline TG mice showed the highest number of entries while the WT mice and most of the suramin treated TG mice showed a similar number of entries.

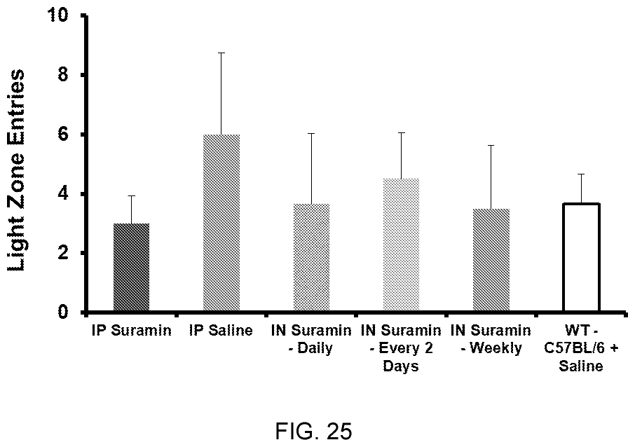

FIG. 25 shows the total number of Light Zone Entries per treatment group.

### Discussion:

Dark Zone Entry Latency: There were substantial and significant differences in Dark Zone entry latencies between the training day and test day in both WT and TG mice treatment groups. This suggests that all experimental groups can learn and remember the footshock-induced fear responses.
Light Zone Total Time and Percentage of Time in the Light Zone: The amount of time spent in the light zone, both total time (minutes) and the percentage of time spent in the light zone relative to time spent in the dark zone (%), provides data regarding each treatment group's general behavior activity and activity levels and rule out potential false positive effects.

Most TG mice treatment groups displayed similar behavior to the WT Control group. This suggests the treatments in these TG mice did not have a significant negative effect on their learning and associative memory.

Number of Dark Zone Entries: The number of dark zone entries reflects the mouse's conditioned fear of a mild foot shock associated with the Dark Zone. Once they enter the dark box, the number of re-entries back into the dark zone is an indicator of how much retained fear they have of entering the dark box. The WT and TG treatment groups with suramin and other comparators show fewer Dark Zone entries compared with the IP saline TG mice suggesting that they have an improved memory from the previous day's conditioning.

### Conclusion:

The results from the Step-Through Passive Avoidance Test suggest that all treatment groups have an intact short-term memory, can learn to avoid the negative stimulus associated with the Dark Zone and retain this conditioned memory for at least 24 hours.

Incorporation by Reference

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

Equivalents

The invention can be embodied in other specific forms without departing from the spirit or essential characteristics thereof. The foregoing embodiments are to be considered in all respects illustrative rather than limiting on the invention described herein. In the various embodiments of the methods and compositions of the present invention, where the term comprises is used with respect to the recited steps of the methods or components of the compositions, it is also contemplated that the methods and compositions consist essentially of, or consist of, the recited steps or components. Furthermore, the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

Furthermore, it should be recognized that in certain instances a composition can be described as composed of the components prior to mixing, because upon mixing certain components can further react or be transformed into additional materials.

All percentages and ratios used herein, unless otherwise indicated, are by weight. It is recognized the mass of an object is often referred to as its weight in everyday usage and for most common scientific purposes, but that mass technically refers to the amount of matter of an object, whereas weight refers to the force experienced by an object due to gravity. Also, in common usage the “weight” (mass) of an object is what one determines when one “weighs” (masses) an object on a scale or balance.

1. A method of treating a nervous system disorder in a human patient in need thereof, comprising intranasally administering to said patient a pharmaceutical composition comprising a therapeutically effective amount of suramin, or a pharmaceutically acceptable salt, ester, solvate, or prodrug thereof, wherein said composition provides an improvement in said patient in at least one of the following disorders, symptoms, or behavioral manifestations of the nervous disorder:
a) anxiety or anxiety-like behavior,
b) willingness to explore the environment,
c) social interaction,
d) spatial learning and memory,
e) learning and memory,
f) irritability, agitation and or crying,
g) lethargy and/or social withdrawal,
h) stereotypic behavior,
i) hyperactivity and/or noncompliance, or
j) restrictive and/or repetitive behaviors.

2.-5. (canceled)

6. A method according to claim 1 wherein said salt is the hexa-sodium salt.

7. A method according to claim 1 wherein the nervous system disorder is selected from autism spectrum disorder (ASD), fragile X syndrome (FXS), fragile X-associated tremor/ataxia syndrome (FXTAS), myalgic encephalomyelitis/chronic fatigue syndrome (ME/CFS), post-traumatic stress syndrome (PTSD), Tourette's syndrome (TS), Parkinson's Disease, Angelman syndrome (AS), and the CNS disorder manifestations often associated with Lyme disease and other tick-borne diseases, and the nervous system and central nervous system (CNS) disorders associated with COVID-19 and other viruses (e.g. Epstein Barr Human Herpesvirus 6 and 7, Herpes Simplex Virus, Cytomegalovirus, and others), including their long term effects.

8. A method according to claim 1 wherein the nervous system disorder is selected from autism spectrum disorder, FXS, or FXTAS.

9. A method according to claim 7 wherein the nervous system disorder is autism spectrum disorder.

10. A method according to claim 9 wherein said autism spectrum disorder is selected from autistic disorder, childhood disintegrative disorder, pervasive developmental disorder-not otherwise specified (PDD-NOS), and Asperger syndrome.

11. A method according to claim 9 wherein said autism spectrum disorder manifests one or more symptoms selected from difficulty communicating, difficulty interacting with others, and repetitive behaviors.

12.-20. (canceled)

21. A method according to claim 1 wherein said composition is administered, at least once daily, or at least twice daily, or at least once weekly, or at least twice weekly, or at least once every two weeks, or at least once monthly, or at least once every 4 weeks.

22. A method according to claim 1 wherein said composition is administered, at least once about every 41 days to about 78 days.

23. A method according to claim 1 wherein said composition is administered, at least once about every 50 days.

24. A method according to claim 1 wherein said composition is administered, at least once per a time interval based on the average half-life of suramin.

25. A method according to claim 1 wherein the composition exhibits, a penetration rate of about 1 micrograms/cm² per hour to about 200 micrograms/cm² per hour of suramin, based on the suramin active, through cultured human airway tissue.

26. A method according to claim 1 wherein the plasma level of the suramin in the patient is maintained at less than about 3 micromolar (μM), or less than about 2.75 micromolar, or less than about 2.5 micromolar, or less than about 2 micromolar, or less than about 1 micromolar, or less than about 0.5 micromolar based on the suramin active.

27. A method according to claim 1 wherein the brain tissue level of the suramin in the patient is from about 1 ng/ml to about 1000 ng/ml.

28. A method according to claim 1 wherein the brain tissue level of the suramin in the patient is at least about 1 ng/ml, or at least about 10 ng/ml, or at least about 50 ng/ml, or at least about 100 ng/ml, or at least about 250 ng/ml, or at least about 500 ng/ml.

29. A method according to claim 1 wherein the brain tissue to blood plasma partitioning ratio for the suramin is at least about 0.05, or at least about 0.1, or at least about 0.25, or at least about 0.50.

30. A method according to claim 1 wherein the AUC for the plasma level for the suramin active for the patient is less than about 80 μg*day/L or is less than about 75 μg*day/L, or is less than about 50 μg*day/L, or is less than about 25 μg*day/L, or is less than about 10 μg*day/L.

31. A method according to claim 1 wherein the Cₘₐₓ for the plasma level for the suramin active for the patient is less than about 75 micromolar, or is less than about 7.5 micromolar, or is less than about 0.1 micromolar, and optionally at least about 0.01 micromolar, based on a single dose.

32. A method according to claim 8 wherein treating said autism spectrum disorder, FXS, or FXTAS comprises improving one or more symptoms of said patient relative to symptoms of said patient prior to said administration, wherein said one or more symptoms are selected from difficulty communicating, difficulty interacting with others, and repetitive behaviors.

33. A method according to claim 8 wherein treating said autism spectrum disorder, FXS, or FXTAS comprises improving an assessment score of said patient relative to a score from said patient prior to said administration.

34. A method according to claim 33 wherein the assessment score is selected from ABC, ADOS, ATEC, CARS CGI, and SRS.

35. A method according to claim 1 wherein the composition is a nasal spray.

36.-38. (canceled)

39. A method of treating a nervous system disorder in a human patient in need thereof, comprising intranasally administering to said patient a pharmaceutical composition comprising an effective amount of suramin, or a pharmaceutically acceptable salt, ester, solvate, or prodrug thereof, wherein said composition, when evaluated in a transgenic FMR mouse model, provides an improvement in at least one of the following behavioral manifestations:
a) light/dark test (LDT),
b) locomotor activity test,
c) social interaction test,
d) Morris Water Maze Test (MWM), or
e) step through passive avoidance test.

40.-41. (canceled)

42. A device for performing the method of claim 1, comprising a nasal spray inhaler for intranasally administering said pharmaceutical composition.
